SEQUENCE LISTING (8 sequences)

SEQ ID NO: 1 - DNA, 104 bp, synthetic construct
  source 1..104: mol_type = other DNA
tttttcgtgcagtacgcaacctttctcatgcgctgcccctcttaaaaaaaagcagatcctaagccgcacccaattaaggggcagcgcatgagaaaggttccgta

SEQ ID NO: 2 - DNA, 24 bp, synthetic construct
  source 1..24: mol_type = other DNA
gcagatcctaagccgcacccaatt

SEQ ID NO: 3 - DNA, 87 bp, synthetic construct
  source 1..87: mol_type = other DNA
aattgggtgcggcttaggatctgcacaaaggcagatcctaagccgcactgggaagggagggtgggaagggagggtgggaagggaggg

SEQ ID NO: 4 - DNA, 67 bp, synthetic construct
  source 1..67: mol_type = other DNA
gcagatcctaagccgcacccaattgtgcggcttaggatctgcctttgtagaagaaggtgtttaagta

SEQ ID NO: 5 - DNA, 55 bp, synthetic construct
  source 1..55: mol_type = other DNA
agggcgggtgggtgtttaagttggagaattgtacttaaacaccttcttcttgggt

SEQ ID NO: 6 - DNA, 41 bp, synthetic construct
  source 1..41: mol_type = other DNA
tgggtcaattctccaacttaaactagaagaaggtgtttaag

SEQ ID NO: 7 - DNA, 48 bp, synthetic construct
  source 1..48: mol_type = other DNA
aattgggtgcggcttaggatctgcacaaaggcagatcctaagccgcac

SEQ ID NO: 8 - DNA, 48 bp, synthetic construct
  source 1..48: mol_type = other DNA
gcagatcctaagccgcacccaattgtgcggcttaggatctgcctttgt

(19) United States
(12) Patent Application Publication
(10) Pub. No.: US 2025/0034621 A1
(43) Pub. Date: Jan. 30, 2025

(54) LOCKED APTAMER HAIRPIN INTEGRATED HYBRIDIZATION CHAIN REACTION FOR RAPID, INSTRUMENT-FREE BIOMARKER DETECTION

(71) Applicant: The Texas A&M University System, College Station, TX (US)

(72) Inventors: Sayantan TRIPATHY, College Station, TX (US); Samuel MABBOTT, Bryan, TX (US); Gerard L. COTE, College Station, TX (US)

(21) Appl. No.: 18/782,100

(22) Filed: Jul. 24, 2024

Related U.S. Application Data
(60) Provisional application No. 63/515,924, filed on Jul. 27, 2023.

(51) Int. Cl. C12Q 1/682; G01N 33/543; G01N 33/68

(52) U.S. Cl. CPC C12Q 1/682; G01N 33/54326; G01N 33/54346; G01N 33/6887; G01N 2800/324

(57) ABSTRACT

In one aspect, the disclosure relates to a universal method for biomarker extraction and detection from clinical samples using magnetic nanoparticles (MNPs) coupled with a locked aptamer-initiator approach. The MNP bound locked aptamer undergoes a conformational change in the presence of the target biomarker and the unfolded initiator triggers a hybridization chain reaction with a structurally stabilized catalytic trimeric triplex DNAzyme hairpin. Amplicons from the hybridization chain reaction can be incubated with hemin to obtain a stable triplex structure that has horseradish peroxidase-like activity in the presence of H₂O₂ and a substrate molecule. The intensity of the signal is directly proportional to the amount of biomarker present. Binding one or more reagents on a nanoparticle can be coupled with this approach for use in a microfluidic or paper fluidic platform. In any of these aspects, the assay can detect as low as femtogram amounts of biomarker from the clinical sample.

### CROSS REFERENCE TO RELATED APPLICATIONS

This application claims the benefit of U.S. Provisional Application No. 63/515,924 filed on Jul. 27, 2023, which is incorporated herein by reference in its entirety.

### STATEMENT REGARDING FEDERALLY SPONSORED RESEARCH OR DEVELOPMENT

This invention was made with Government support under grant number ERC (PATHS-UP) 1648451, awarded by the National Science Foundation. The government has certain rights in the invention.

### CROSS REFERENCE TO SEQUENCE LISTING

This application contains a sequence listing filed in ST.26 format entitled “922001-1130_Sequence_Listing.xml” created on Jul. 12, 2024, and having a file size of 8,514 bytes. The content of the sequence listing is incorporated herein in its entirety.

### BACKGROUND

Detection of biomarkers is useful as a non-invasive way to diagnose and monitor the course of diseases and treatments thereof. However, current biomarker identification and quantitation methods are typically conducted at a research and development scale where a non-enzymatic hybridization chain reaction is often integrated with high-cost analytical systems such as fluorescence microscopes and flow cytometry. Several hybridization chain reaction-based diagnostics have been reported, but these suffer from nonspecific readout generation based on other molecules in the clinical samples, and are not practical for point of care use. In many cases, the readouts for existing techniques are based on the quantitative polymerase chain reaction or enzyme-based isothermal amplification which needs a centralized lab and highly trained personnel. Furthermore, for many cases, prior nucleic acid or biomarker extraction is required for the assay to be successful.

Despite advances in biomarker detection research, there is still a scarcity of methods for detecting and/or quantifying biomarkers that are easy to use, low in cost, portable, specific, and efficient. An ideal detection method and device is one that could be easily and affordably be operated at the point of care, making the method suitable for use in developing countries, remote locations, and in other situations where complicated and expensive equipment may not be practical. An ideal method would be useful for peptide, protein, DNA, and RNA biomarkers, including various small and micro RNA species. These needs and other needs are satisfied by the present disclosure.

### SUMMARY

In accordance with the purpose(s) of the present disclosure, as embodied and broadly described herein, the disclosure, in one aspect, relates to a universal method for developing protein or nucleic acid biomarker extraction followed by detection from clinical samples using magnetic nanoparticles (MNPs) coupled with a locked aptamer-initiator approach. In one aspect, the magnetic nanoparticle bound locked aptamer will undergo a conformational change in the presence of the target biomarker and the unfolded initiator will trigger a hybridization chain reaction with a structurally stabilized catalytic trimeric triplex DNAzyme hairpins. In one exemplary aspect, the amplicons from the hybridization chain reaction can be incubated with hemin to obtain a stable triplex structure that has horseradish peroxidase-like activity in the presence of hydrogen peroxide and specific substrates such as 2,2′-azinobis-(3-ethylbenzothiazoline-6-sulfonic acid) (ABTS) or 3,3′,5,5′-tetramethylbenzidine (TMB). The intensity of color will be directly proportional to the amount of biomarker present. In an alternative aspect, fluorescent, absorption, Raman, electrochemical or other methods for visualizing or recording a signal from the disclosed method are disclosed. In one aspect, the visualization method is instrument-free or can be accomplished using an affordable easy to use portable reader or commonly available electronic devices such as cell phones, tablets, digital cameras, or laptop computer equipment. In any of these aspects, the assay can detect as low as femtogram amounts of biomarker from the clinical sample.

Other systems, methods, features, and advantages of the present disclosure will be or become apparent to one with skill in the art upon examination of the following drawings and detailed description. It is intended that all such additional systems, methods, features, and advantages be included within this description, be within the scope of the present disclosure, and be protected by the accompanying claims. In addition, all optional and preferred features and modifications of the described embodiments are usable in all aspects of the disclosure taught herein. Furthermore, the individual features of the dependent claims, as well as all optional and preferred features and modifications of the described embodiments are combinable and interchangeable with one another.

### BRIEF DESCRIPTION OF THE DRAWINGS

Many aspects of the present disclosure can be better understood with reference to the following drawings. The components in the drawings are not necessarily to scale, emphasis instead being placed upon clearly illustrating the principles of the present disclosure. Moreover, in the drawings, like reference numerals designate corresponding parts throughout the several views.

FIG. 1A shows a TEM image of bare iron oxide. FIG. 1B shows a TEM image of silica-containing MNPs conjugated to streptavidin (SiMNP-Strep). Scale bars are 50 nm.

FIGS. 2A-2C show gel electrophoresis results using different concentrations of secondary aptamer initiator that is not conformationally locked (10 pM to 100 aM). FIG. 2A: Limit of detection determination (target range 100 fM-100 aM) using oligonucleotide set 3 (limit of detection “LoD” 1 fM). FIG. 2B: Limit of detection determination (target range 10 pM-100 aM) using oligonucleotide set 3 (LoD 10 fM). Nonspecific amplification was seen in lane 6 for the structural instability of hairpin 1. FIG. 2C: Mitigation of non-specificity using low salt concentration (5 mM) (no nonspecificity at lanes 7 and 8).

FIGS. 3A-3D show colorimetry optimization using hairpin 1 (H1)-DNAzyme and hybridization chain reaction (HCR) products. FIG. 3A: Stability of color using hemin incubated H1-DNAzyme in 0 hours and 24 hours. FIG. 3B: Optimization of three different H1-DNAzymes from different oligonucleotide set where H1 DNAzyme from oligo set 3 gave the best color intensity. FIG. 3C: HCR using oligo set 3 in presence of 1 pg target followed by colorimetry. 15 minutes of incubation is sufficient to create the chain from which color has been produced. FIG. 3D: RGB intensity calculation using the data of HCR followed by colorimetry in presence of target.

FIG. 4 shows a schematic of a disclosed assay using streptavidin-coated gold nanostars. Step 1, Incubation of cardiac troponin I (cTnl) with locked aptamer. Step 2, Hybridization of biotinylated hairpins with the MNP-aptamer bound cTnl. Step 3, Incubation with Streptavidin coated gold nanostar (Strep-SiAuNS).

FIGS. 5A-5F show structural simulation of oligonucleotides. FIG. 5A: Unmodified aptamer against cardiac troponin I FIG. 5B: Modified aptamer with initiator (structural stability increased) FIG. 5C: Hairpin 1 FIG. 5D: Hairpin 2 FIG. 5E: No cross reaction within the oligonucleotides FIG. 5F: Probable hybridized chain reaction amplicon

FIGS. 6A-6B show NUPACK simulation of the oligonucleotides and gel electrophoresis study. FIG. 6A: Amplicon formation in presence of initiator, hairpin 1 (H1) and hairpin 2 (H2). FIG. 6B: Gel electrophoresis study. Lane 1: 50 bp ladder; Lane 2: 1 ng/mL cTnl+1 μM Aptamer+1 μM H1+1 μM H2 (multiple band formation indicates opening up of multiple aptamers which causes multiple short length chain formation). Lane 3: 100 μg/mL cTnl+Aptamer+1 μM H1+1 μM H2 (band formation at the upper side of gel indicates opening up of less aptamers which causes multiple high length chain formation). Lane 4: 1 pg/mL cTnl+1 μM Aptamer+1 μM H1+1 μM H2 (band formation at the very top side of gel indicates opening up of very less aptamers which causes very long chain formation which could not migrate through the gel properly). Lane 5: Negative Control.

FIGS. 7A-7E show characterization of surface modified gold nanostars. FIG. 7A: TEM image of IR780 dye coupled silica coated gold nanostar (SiAuNS). Average diameter is approximately 100-120 nm. FIG. 7B: UV-Vis spectra of gold nanoparticles (AuNP), gold nanostar (SiAuNS), silica coated gold nanostar (SiAuNS). FIG. 7C: Raman spectra of bare gold nanostar and silica coated gold nanostar (SiAuNS). FIG. 7D: Hydrodynamic size of gold nanoparticles (AuNP), gold nanostar (SiAuNS), silica coated gold nanostar (SiAuNS), FIG. 7E: Zeta potential of all surface modified gold nanostars.

FIGS. 8A-8D show sensitivity of assay determination employing surface-enhanced resonance Raman scattering (SERRS) using cTnl in aqueous buffer solution. FIG. 8A: Design of magnetic concentrator (sample insert and magnetic holder. FIG. 8B: Raman spectra of Streptavidin coated SiAuNS (peak at 1206 cm⁻¹. FIG. 8C: There is prominent peak at 1206 cm⁻¹ when magnetic concentrator was used to concentrate magnetic nanoparticles bound HCR-Streptavidin-SiAuNS complex. Very less Raman peak intensity was observed in case of 384 well plate. FIG. 8D: Assay sensitivity determination using SERRS. The sensitivity up to 50 fg/mL was achieved using cTnl in aqueous buffer solution.

FIG. 9 shows determination of LoD with human serum spiked cardiac troponin I using 1 μM of hairpins. Very much less discrimination was seen in lower concentration (50 pg/mL-500 fg/mL range).

FIGS. 10A-10B show sensitivity of assay determination employing SERRS using cTnl spiked in human serum. FIG. 10A: SERRS spectra obtained from the assay using cTnl spiked in serum. Higher concentration of Streptavidin-SiAuNs and oligo hairpins results higher sensitivity in the assay. FIG. 10B: Standard curve generation from the peak intensity obtained from the assay. A robust linear relationship between Raman intensity and cTnl concentration was observed.

FIGS. 11A-11C show performance metrics for the Random Forest Classifier used to discriminate serum samples according to cTnl levels. FIG. 11A: ROC-AUC (0.93), FIG. 11B: Precision-Recall curve (0.94), FIG. 11C: Confusion matrix (negatives <40 pg/mL cTnl, positives >40 pg/mL).

FIG. 12A shows a schematic of hyperbranched HCR reaction. FIG. 12B shows a colorimetric reaction scheme. FIG. 12C shows a portable imager for red-green-blue (RGB) analysis

FIGS. 13A-13E show results from the hyperbranched colorimetric assay. Briefly, before purchasing the hairpin sequences used to facilitate the hyperbranched HCR amplification, over 100 candidates were screened through simulation studies in NUPACK. This allowed identification of sequences that minimized unwanted cross-hybridization in the absence of the modified Tro4 aptamer and cTnl. FIG. 13A shows simulation results for the top 4 candidates capable of facilitating the hyperbranched HCR amplification mechanism with minimal side product formation FIG. 13B shows fluorescence studies to evaluate whether hairpin HTG-H1 hybridizes with the initiator sequence of the modified Tro4 aptamer-conjugated MPs in the presence of varying concentrations of cTnl, FIG. 13C shows the protocol used for the preliminary colorimetric cTnl testing studies. Briefly, the aptamer-conjugated magnetic particles (MPs) and hairpins (a combined volume of 25 μL in a pre-optimized high salt concentration assay buffer) are mixed with the cTnl sample (25 μL) in a sample tube. This mixture is then placed onto the sample platform of the imaging system and incubated for 15 minutes. After incubation, the MPs are magnetically separated, and the supernatant is removed and replaced with ABTS²⁻/H₂O₂ solution. Imaging begins immediately at this stage. The magnet is removed to allow the MPs to disperse within the ABTS²⁻/H₂O₂ solution, increasing the surface area for catalysis. A 5-minute dispersion period was found to provide the optimal balance between assay time and the differentiation of cTnl at low concentrations. The magnet is then reapplied, pulling the MPs aside to allow the extent of the green color change to be observed. Imaging continues for an additional 5 minutes. FIG. 13D shows an example real-time time-series plot displayed on the graphical user interface (GUI) shows the change in the averaged G channel value extracted from images collected every 30 seconds over a 10-minute period. FIG. 13E shows Δ_{Greyscale} values are subtracted from 255 to generate a direct relationship across a wide dynamic range of cTnl concentrations.

FIG. 14 shows colorimetry experiments using different concentrations of cardiac troponin I.

FIGS. 15A-15C show RGB analysis using Python code extracted RGB values from the portable colorimetric imaging system shown in FIG. 12C.

FIGS. 16A-16B show schematics of two related proposed methods for biosensing.

Additional advantages of the invention will be set forth in part in the description which follows, and in part will be obvious from the description, or can be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### DETAILED DESCRIPTION

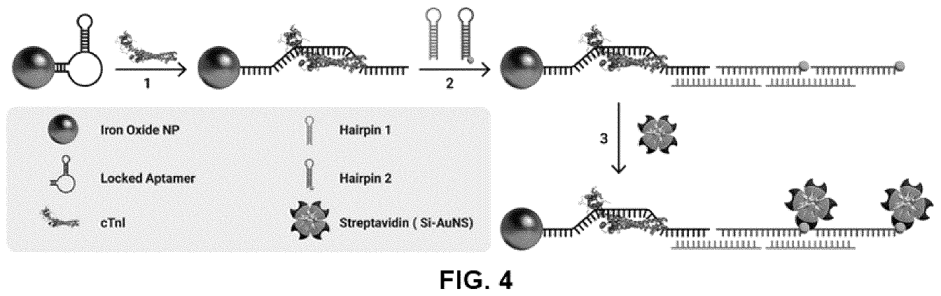

Disclosed herein is a universal method for developing protein or nucleic acid biomarker extraction followed by detection from clinical samples using magnetic nanoparticles (MNPs or MPs) coupled with a locked aptamer-initiator approach which performs with higher accuracy and precision than known sandwich aptamer methods. The magnetic nanoparticle bound locked aptamer undergoes a conformational change in the presence of the target and the unfolded initiator will trigger the hybridization chain reaction with structurally stabilized catalytic trimeric triplex DNAzyme hairpins. The amplicons from the hybridization chain reaction are incubated with hemin to obtain a stable triplex structure that has horseradish peroxidase-like activity in the presence of hydrogen peroxide and specific substrates such as 2,2′-azinobis-(3-ethylbenzothiazoline-6-sulfonic acid) (ABTS). The intensity of color is directly proportional to the amount of biomarker present (FIGS. 4, 12A, and 16A-16B). The assay can detect as low as femtogram amounts of biomarker from the clinical sample.

In one aspect, the method can be used at the point of care (POC) and can detect biomarkers including proteins, nucleic acids, and/or viable pathogens. Detection can be accomplished colorimetrically and/or via other methods depending upon available equipment.

The disclosed method includes a biotinylated aptamer-initiator (initiator of downstream hybridization chain reaction) for a specific target that is locked with a blocker sequence by incorporating an initiator and partial complementary aptamer sequence at the 3′ end of the aptamer. The structurally stabilized locked aptamer undergoes the conformational change in the presence of a specific target. As a result, the initiator with the blocker sequence is released. The released initiator triggers the hybridization chain reaction with two metastable hairpins employing toehold-mediated strand displacement. Hairpin 1 includes a trimeric DNAzyme sequence in the loop of the hairpin which will create a triplex structure. The triplex structure includes guanine-rich sequences which generally form higher-order structures and fold into a parallel or an antiparallel G-triplex in the presence of potassium salts. This trimeric triplex binds to hemin subunits, which have horseradish peroxidase activity in the presence of hydrogen peroxide, and specific color-generating substrates such as ABTS. The absence of a target results in the closing of locked aptamer and thus amplification followed by color generation will not take place. This method can specifically detect any biomarker from a clinical sample using basic, readily-available equipment in a cost-efficient manner. Personnel training is minimal in order to use the method, and it does not require washing steps, which typically introduce manual error for currently known methods. In the disclosed method, multiple samples can be probed simultaneously. In one aspect, currently-used methods require nucleic acid or biomarker extraction in order to successfully perform assays, whereas the present method does not require those steps.

In one aspect, the instrumentation and reagents required to perform the method are portable. In a further aspect, color generation using trimeric triplex catalytic DNAzyme can be monitored with a lab-built portable imager coupled with straightforward Python code which gives proper RGB analysis of color generated from the sample. In an alternative aspect, fluorescence, absorption, Raman, electrochemical, and/or other methods for visualizing or recording a signal from the disclosed method can be used. In a further aspect, the one or more reagents can be bound onto nanoparticles coupled with a locked aptamer-initiator approach that flow down a paper fluidic or microfluidic platform creating a bound section within the fluidic device that produces a detectable color, absorption, fluorescence, and/or Raman signals.

In one aspect, the assay includes a hybridization chain reaction, which is an enzyme-free method and, unlike gold standard procedures such as polymerase chain reaction, it does not require multiple temperature-based annealing steps. In one aspect, the method can be performed at room temperature.

In another aspect, the method can quickly and effectively produce reliable colorimetric readouts with reduced error. In an aspect, the locked aptamer makes the assay specific to selected targets, while magnetic nanoparticles can efficiently remove the other contaminants in clinical samples.

In an aspect, the method satisfies a demand for highly efficient and cost-effective molecular diagnostic assays. In another aspect, the method can be used by research and development organizations, industrial manufacturers, academic institutions, government agencies, pharmacy companies, pathology labs, rural hospitals, and in developing countries where higher-cost methods may not be available. In some aspects, the method can be performed without using any instruments requiring electric power.

In any of these aspects, the method can be used to detect biomarkers such as, for example, cardiac troponin I, at the femtomolar level.

Disclosed herein is a method for detecting a biomarker in a clinical sample, the method including at least the steps of:
(a) binding an aptamer locked in a first conformation to a magnetic nanoparticle;
(b) contacting the magnetic nanoparticle and aptamer with a clinical sample;
wherein presence of the biomarker in the clinical sample causes aptamer to undergo a conformational change to a second conformation;
wherein the conformational change results the opening of an initiator which triggers a hybridization chain reaction with a trimeric triplex DNAzyme sequence incorporating a first hairpin and a second hairpin, wherein the first hairpin and the second hairpin are partially complementary to each other, forming a structurally stabilized catalytic trimeric triplex;
(c) contacting the structurally stabilized catalytic trimeric triplex with one or more reagents to produce a detectable signal; and
(d) detecting the signal.

In one aspect, the biomarker can be a peptide, a protein, DNA, RNA, carbohydrate, lipids a bacterium, a parasite, or a virus. In an aspect, the biomarker can be cardiac troponin I. In one aspect, when the biomarker is cardiac troponin I, the aptamer can have SEQ ID NO. 1, the first hairpin can have SEQ ID NO. 7, and the second hairpin can have SEQ ID NO. 8. In some aspects, the method can further include contacting the magnetic nanoparticle, aptamer, and clinical sample with a third hairpin and a fourth hairpin. In a further aspect, incorporation of the third and fourth hairpins can result in forming a hyperbranched chain connected to the magnetic nanoparticle, wherein the hyperbranched chain results in an increased signal intensity relative to an unbranched chain. In an aspect, and without wishing to be bound by theory, more molecules responsible for generating a signal (e.g. biotin for binding to streptavidin coated gold particles, or separated fluorophores and quenchers) can be incorporated into the hyperbranched chain. In an aspect, when the biomarker is cardiac troponin I, the first hairpin can have SEQ ID NO. 4, the second hairpin can have SEQ ID NO. 6, the third hairpin can have SEQ ID NO. 3, and the fourth hairpin can have SEQ ID NO. 5.

In any of these aspects, the magnetic nanoparticle can be or include iron oxide (Fe₃O₄). In one aspect, the iron oxide is co-precipitated with silica, and the silica can be amino functionalized before or after precipitation using (3-aminopropyl)triethoxysilane (APTES). In some aspects, the magnetic nanoparticle has a streptavidin coating. In a further aspect, the streptavidin coating is conjugated to the APTES. In still another aspect, the aptamer is conjugated to biotin, and the biotin binds to the streptavidin coating, thereby coating the magnetic nanoparticle with the aptamer.

In some aspects, the one or more reagents can include hemin, hydrogen peroxide, 3,3′,5,5′-tetramethylbenzidine (TMB), ABTS (2,2′-azino-bis(3-ethylbenzothiazoline-6-sulfonic acid)) or any combination thereof, and a substrate, and wherein the hemin induces stabilization of the catalytic trimeric triplex, wherein the stabilized catalytic trimeric triplex has horseradish peroxidase-like activity in the presence of the hydrogen peroxide and the substrate.

In another aspect, the detectable signal can be a fluorescence signal, a color or other optical signal, an electrochemical signal, a Raman signal, or another signal. In a further aspect, when the signal is a fluorescence signal, fluorescence can be generated when at least one component is conjugated to a fluorescence marker such as, for example, FAM or Cy5. In some aspects, one or more of the disclosed hairpins include a fluorophore and a quencher and produce no signal when the hairpins are closed/base paired to themselves, but when the hairpins open in the presence of the biomarker, the fluorophores are no longer quenched and can produce a signal.

In a further aspect, when the detectable signal is a color, the intensity of the color or the rate of change of color can be correlated to the amount of biomarker present, such that a darker color with time is indicative of a higher amount of biomarker, while a lighter color is indicative of a lower amount of biomarker. Further in this aspect, the substrate can be 2,2′-azinobis-(3-ethylbenzothiazoline-6-sulfonic acid) (ABTS), and the horseradish peroxidase-like activity produces a color in the presence of the hydrogen peroxide and the ABTS.

In any of these aspects, the method can be instrument-free as defined elsewhere herein, or does not require the use of specialized electronic instruments to perform. In a further aspect, an instrument-free method may still make use of a commonly-available device such as, for example, a smartphone, tablet, or laptop computer for interpreting and quantifying signals.

In another aspect, the one or more reagents can be bound or include a gold particle such as a gold nanosphere or gold nanostar and the detectable signal can be or include fluorescent or Raman scattering. In an aspect, a gold nanostar as described herein can have a higher surface area, which leads to more reactivity for conjugation to dyes or other reporter molecules and thus a higher signal, even from a smaller sample size. In some aspects, the gold particle includes a silica shell. In some aspects, the gold particle includes a plurality of dye molecules, where the dye molecules are encapsulated in the silica shell adjacent to the gold particle surface to the Si—OH groups. In an aspect, the dye can be an NIR dye such as, for example, NIR-780.

In one aspect, the method has a limit of detection in the femtomolar range such as about 10 femtomolar, or about 1 femtomolar. In one aspect, a colorimetric sample holder contains a single magnet that attracts the magnetic particles, allowing color changes in the solution to be more easily observed. In another aspect, a sample holder for the SERRS measurements the sample holder contains 5 magnets (4 on the sides, and 1 on the bottom). In a further aspect, the magnets on the sides are oriented to so they exhibit magnetic repulsion while the magnet on the bottom exhibits attraction. Without wishing to be bound by theory, this enables the nanoparticle deposit at the bottom of the sample well to form a condensed circular pattern which is more amenable for SERRS sampling.

Also disclosed herein is use of the disclosed method for identifying myocardial infarction, wherein the biomarker is cardiac troponin I. In a further aspect, the myocardial infarction can be asymptomatic. However, the disclosed method should be considered to be universally adaptable to any disease or condition where a peptide, protein, DNA, or RNA biomarker can be identified in a clinical sample from a subject.

In still another aspect, when the signal is an electrochemical signal, a redox reporter molecule such as, for example, methylene blue can be used.

In a further aspect the one or more reagents can be bound onto nanoparticles coupled with a locked aptamer-initiator approach that flow down a paper fluidic or microfluidic platform creating a bound section that produces detectable color, absorption, fluorescent or Raman signals. In an aspect, the method can be performed at room temperature.

Many modifications and other embodiments disclosed herein will come to mind to one skilled in the art to which the disclosed compositions and methods pertain having the benefit of the teachings presented in the foregoing descriptions and the associated drawings. Therefore, it is to be understood that the disclosures are not to be limited to the specific embodiments disclosed and that modifications and other embodiments are intended to be included within the scope of the appended claims. The skilled artisan will recognize many variants and adaptations of the aspects described herein. These variants and adaptations are intended to be included in the teachings of this disclosure and to be encompassed by the claims herein.

Although specific terms are employed herein, they are used in a generic and descriptive sense only and not for purposes of limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure.

Any recited method can be carried out in the order of events recited or in any other order that is logically possible. That is, unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided herein can be different from the actual publication dates, which can require independent confirmation.

While aspects of the present disclosure can be described and claimed in a particular statutory class, such as the system statutory class, this is for convenience only and one of skill in the art will understand that each aspect of the present disclosure can be described and claimed in any statutory class.

It is also to be understood that the terminology used herein is for the purpose of describing particular aspects only and is not intended to be limiting. Unless defined otherwise, all technical and scientific terms used herein have the same meaning as commonly understood by one of ordinary skill in the art to which the disclosed compositions and methods belong. It will be further understood that terms, such as those defined in commonly used dictionaries, should be interpreted as having a meaning that is consistent with their meaning in the context of the specification and relevant art and should not be interpreted in an idealized or overly formal sense unless expressly defined herein.

Prior to describing the various aspects of the present disclosure, the following definitions are provided and should be used unless otherwise indicated. Additional terms may be defined elsewhere in the present disclosure.

### Definitions

As used herein, “comprising” is to be interpreted as specifying the presence of the stated features, integers, steps, or components as referred to, but does not preclude the presence or addition of one or more features, integers, steps, or components, or groups thereof. Moreover, each of the terms “by”, “comprising,” “comprises”, “comprised of,” “including,” “includes,” “included,” “involving,” “involves,” “involved,” and “such as” are used in their open, non-limiting sense and may be used interchangeably. Further, the term “comprising” is intended to include examples and aspects encompassed by the terms “consisting essentially of” and “consisting of.” Similarly, the term “consisting essentially of” is intended to include examples encompassed by the term “consisting of.

As used in the specification and the appended claims, the singular forms “a,” “an” and “the” include plural referents unless the context clearly dictates otherwise. Thus, for example, reference to “a detection method,” “a biomarker,” or “an aptamer,” include, but are not limited to, mixtures, combinations, or series of two or more such biomarkers, detection methods, or aptamers, and the like.

It should be noted that ratios, concentrations, amounts, and other numerical data can be expressed herein in a range format. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about,” it will be understood that the particular value forms a further aspect. For example, if the value “about 10” is disclosed, then “10” is also disclosed.

When a range is expressed, a further aspect includes from the one particular value and/or to the other particular value. For example, where the stated range includes one or both of the limits, ranges excluding either or both of those included limits are also included in the disclosure, e.g. the phrase “x to y” includes the range from ‘x’ to ‘y’ as well as the range greater than ‘x’ and less than ‘y’. The range can also be expressed as an upper limit, e.g. ‘about x, y, z, or less' and should be interpreted to include the specific ranges of ‘about x’, ‘about y’, and ‘about z’ as well as the ranges of ‘less than x’, less than y’, and ‘less than z’. Likewise, the phrase ‘about x, y, z, or greater’ should be interpreted to include the specific ranges of ‘about x’, ‘about y’, and ‘about z’ as well as the ranges of ‘greater than x’, greater than y’, and ‘greater than z’. In addition, the phrase “about ‘x’ to ‘y”’, where ‘x’ and ‘y’ are numerical values, includes “about ‘x’ to about ‘y”’.

It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range.

As used herein, the terms “about,” “approximate,” “at or about,” and “substantially” mean that the amount or value in question can be the exact value or a value that provides equivalent results or effects as recited in the claims or taught herein. That is, it is understood that amounts, sizes, formulations, parameters, and other quantities and characteristics are not and need not be exact, but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In such cases, it is generally understood, as used herein, that “about” and “at or about” mean the nominal value indicated ±10% variation unless otherwise indicated or inferred. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise.

As used herein, the terms “optional” or “optionally” means that the subsequently described event or circumstance can or cannot occur, and that the description includes instances where said event or circumstance occurs and instances where it does not.

As used herein, “instrument-free” refers to the condition wherein an expensive piece of analytical equipment (e.g. refrigerated centrifuge, flow cytometer, fluorescence microscope, or the like) is not required in order to qualitatively and/or quantitatively identify the presence and amount of a biomarker in a sample. “Instrument-free” methods as described herein may make use of simple, commonly-available electronic devices such as, for example, smartphones or digital cameras, tablet computers, or laptop computers for analyzing a signal (e.g. for capturing color intensity during a color change reaction).

Unless otherwise specified, temperatures referred to herein are based on atmospheric pressure (i.e. one atmosphere).

Now having described the aspects of the present disclosure, in general, the following Examples describe some additional aspects of the present disclosure. While aspects of the present disclosure are described in connection with the following examples and the corresponding text and figures, there is no intent to limit aspects of the present disclosure to this description. On the contrary, the intent is to cover all alternatives, modifications, and equivalents included within the spirit and scope of the present disclosure.

### EXAMPLES

The following examples are put forth so as to provide those of ordinary skill in the art with a complete disclosure and description of how the compounds, compositions, articles, devices and/or methods claimed herein are made and evaluated, and are intended to be purely exemplary of the disclosure and are not intended to limit the scope of what the inventors regard as their disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### Example 1: Femtomolar Detection of Cardiac Troponin I Using Colorimetry

### Materials and Methods

Instruments and Reagents. All reagents were purchased from Sigma-Aldrich (St. Louis, MO, USA). Hairpin 1 with DNAzyme (H1-DNAzyme), Hairpin 2 (H2), and aptamer trigger/initiator were ordered from IDT (Coralville, IA, USA). The oligos were dissolved in TE buffer, heated to 95° C. for 5 min, cooled quickly in ice (snap-cooled), and then stored at 4° C. A hemin stock solution (10 mM) was prepared in DMSO and stored in the dark at −20° C. 3,3′,5,5′-tetramethylbenzidine (TMB) working solutions were freshly prepared with 0.15 M citrate phosphate buffer pH-5.5. Hemin working solutions were freshly prepared with colorimetric Tris-HCl buffer (25 mM, pH 7.6) containing 150 mM NaCl, 20 mM KCl, 0.03% TritonX-100, and 1% DMSO.

Magnetic Nanoparticle Synthesis, Surface Modification, and Characterization. Iron oxide magnetic nanoparticles (MNPs) were synthesized using a co-precipitation method coated with silica and then amino functionalized using APTES (SiMNP-NH2). Streptavidin is conjugated to the SiMNP-NH2 particles using EDC/NHS chemistry to produce streptavidin functionalized SiMNPs (SiMNP-Strep).

HCR Optimization and Gel Electrophoresis. Different volumes of snap-cooled secondary aptamer-initiator (final concentration of 100 pM to 100 aM), H1-DNAzyme (final concentration 1 μM), H2 (final concentration 1 μM) were mixed to a final volume of 30 μL in sodium phosphate sodium chloride buffer (HCR buffer) and incubated at room temperature for 2 hours. 10 μL of the reaction mixture was loaded in 12% native PAGE. Water was used in place of an aptamer initiator for negative control.

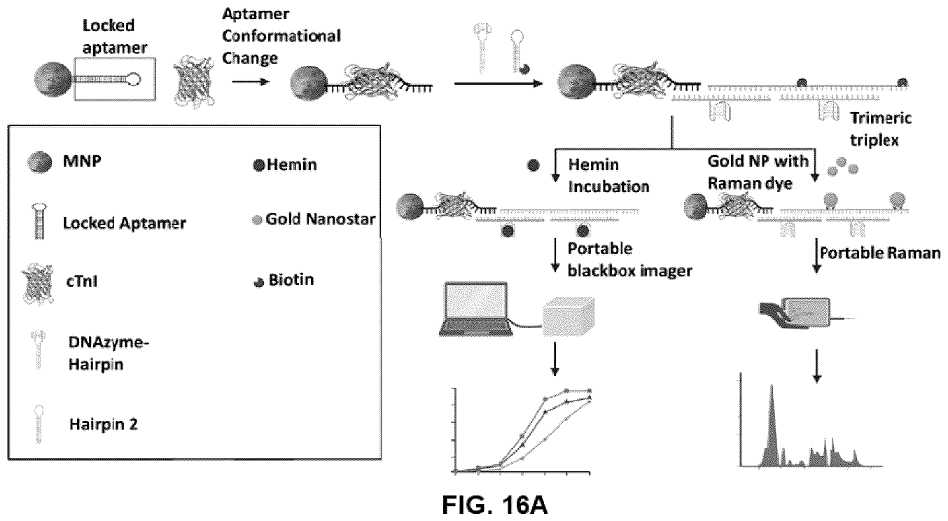

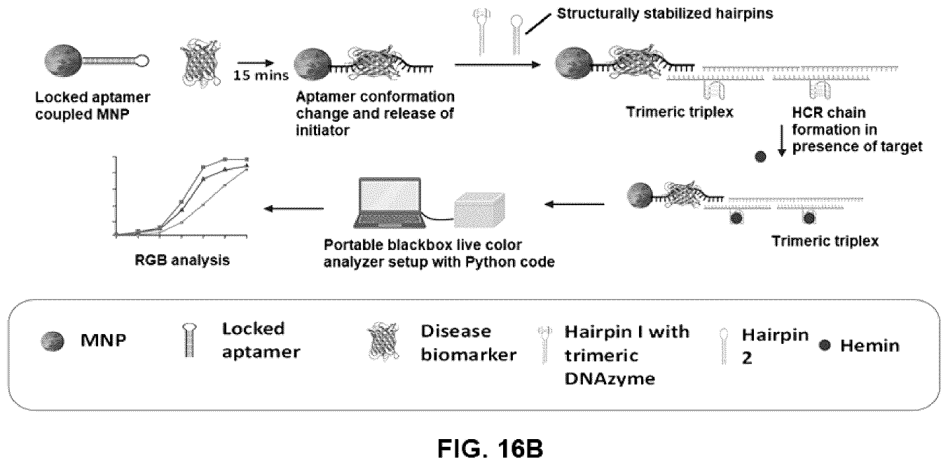

Assay with Cardiac Troponin I. 200 μg of SiMNP-Strep were washed with 1×PBS-T buffer. 1 μM of biotinylated primary aptamer (tro 6) was heated to 95° C. for 5 min, cooled quickly in ice, added to the MNP, and incubated for 15 minutes. For the negative control, water was used in place of a biotinylated aptamer. After 2 magnetic decantation steps using PBS-T buffer, blocking was accomplished using 1 μM biotin after 30 minutes incubation. 1 μM of secondary aptamer was incubated with different concentrations (5 ng/μL to 5 fg/μL) of cardiac troponin I in PBS for 30 minutes. After incubating primary aptamer-bound MNP with secondary aptamer-bound cardiac troponin 1 for 30 minutes, hemin incorporated hairpin 1 and hairpin 2 was added into the solution. After 30 minutes of incubation, magnetic decantation was done by colorimetric buffer for 2 times. In the end, TMB and H₂O₂ were added and color was monitored. The assay scheme is shown in FIG. 16A.

### Results and Discussion

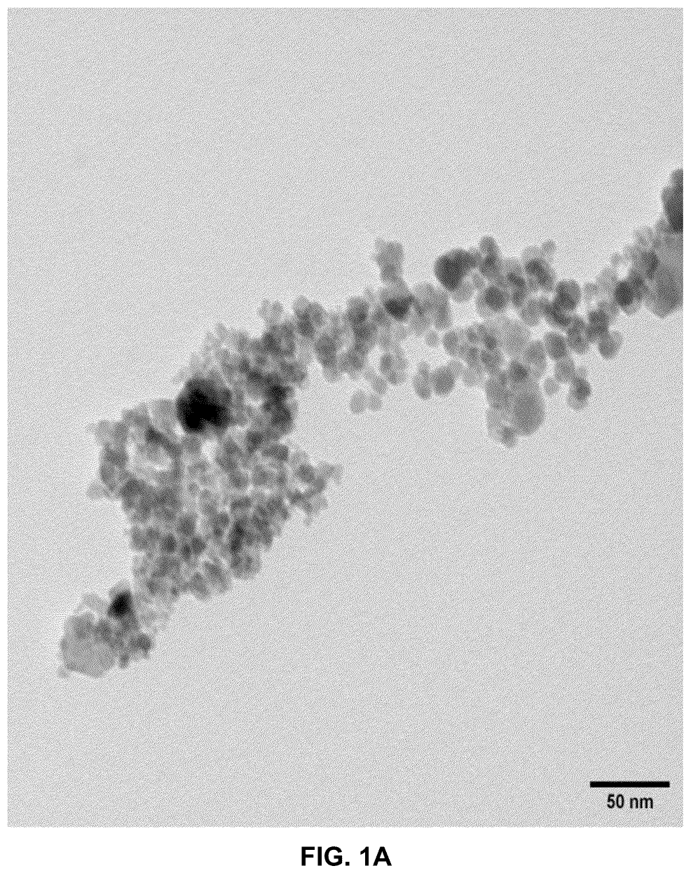

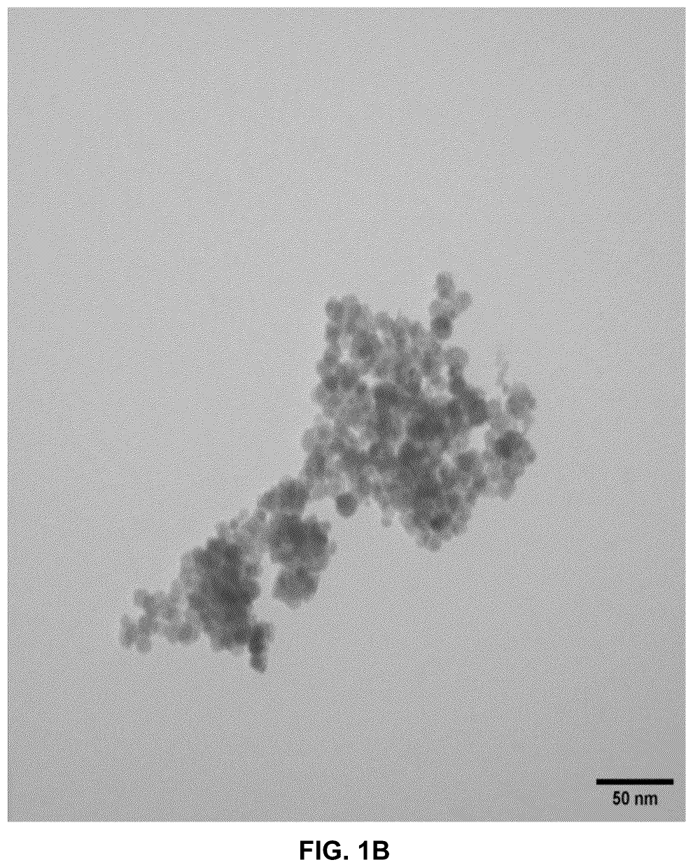

Principle of the Assay and Characterization of Nanoparticles. Biotinylated tro-6 was conjugated to SiMNP-Strep. Secondary aptamer with initiator was incubated firstly with different concentrations of cardiac troponin I and then attached to the MNP-bound primary aptamer to reduce washing steps and non-specific removal of aptamer target complex. After removing unbound residual aptamers by magnetic decantation, structurally stabilized hemin-incubated hairpins were incubated with the MNP-bound aptamer-target complex. After washing, TMB and H₂O₂ were added to obtain the final color. TEM images confirmed that the bare MNP and the streptavidin-coated MNP had an average diameter of 9.91 nm and 10.23 nm respectively (FIGS. 1A-1B). Zeta potential data showed that after modification the surface charge has been changed from −2.3 mV to −21.1 mV which confirms the successful coating of streptavidin on MNP.

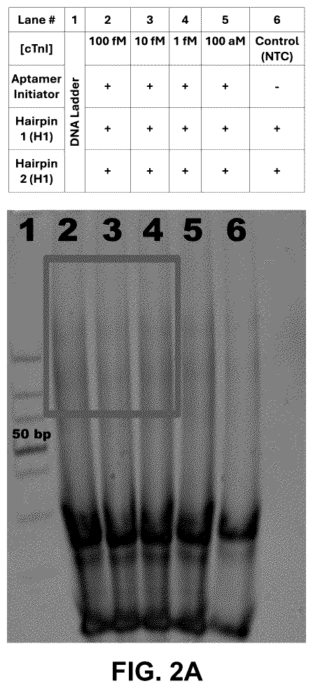

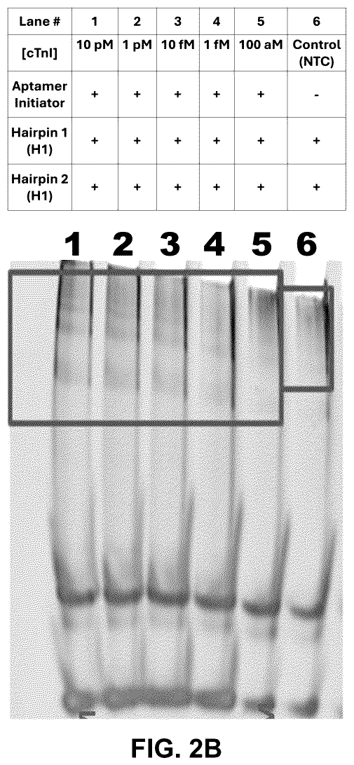

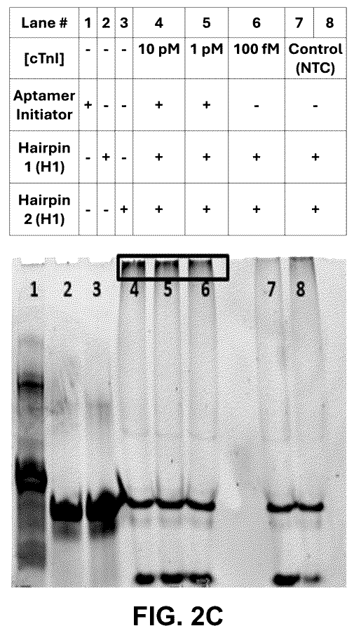

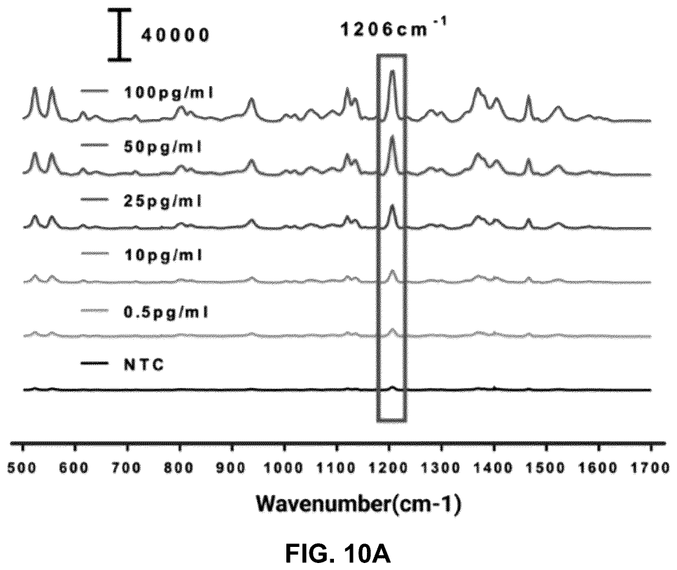

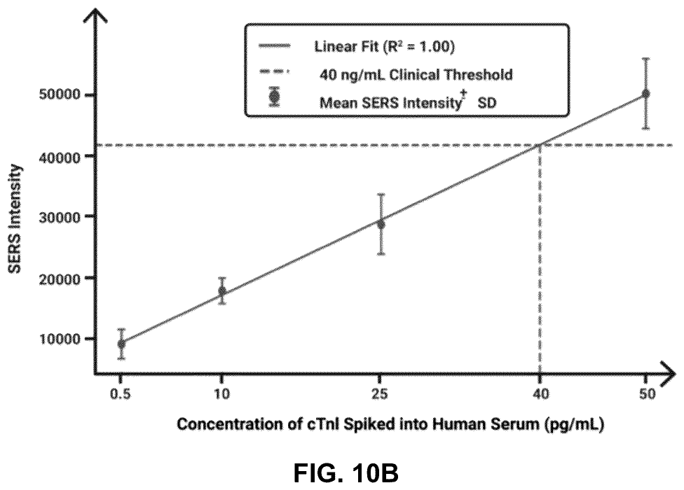

Optimization of HCR Products. The folded structure of the modelled hairpins were verified using 12% Native PAGE. Although all the hairpins were successfully folded, oligonucleotide sets 2 and 3 showed the better high molecular weight band in PAGE than oligonucleotide set 1 in presence of secondary aptamer initiator which binds with the target in actual assay. After The HCR study utilizing a secondary aptamer was done to investigate the nonspecificity. Different concentrations of aptamer initiator (10 pM to 100 aM) was used to investigate the formation of a gel band at high molecular weight to determine hybridization has successfully occurred upon the opening of the meta-stable hairpins. High molecular weight band amplicons were seen for oligo set 2 and oligo set 3 in the presence of 1 fM (FIG. 2A) and 10 fM (FIG. 2B) of initiator. However, a few non-specific amplicons were seen in negative control where no target was present (FIG. 2B, Lane 6). To address this issue, the stability of H1 was increased by introducing a DNAzyme quadruplex to the 3′ end and explored different concentrations of potassium salt in the HCR buffer (100 mM to 20 mM). Although improved specificity was observed (FIG. 2C) after these changes, the nonspecific amplicons could not be completely eliminated.

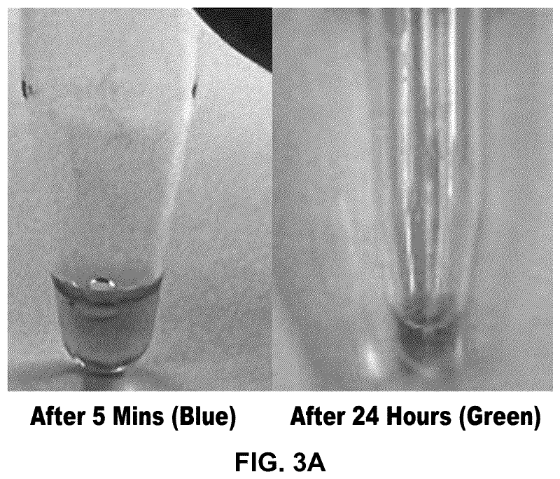

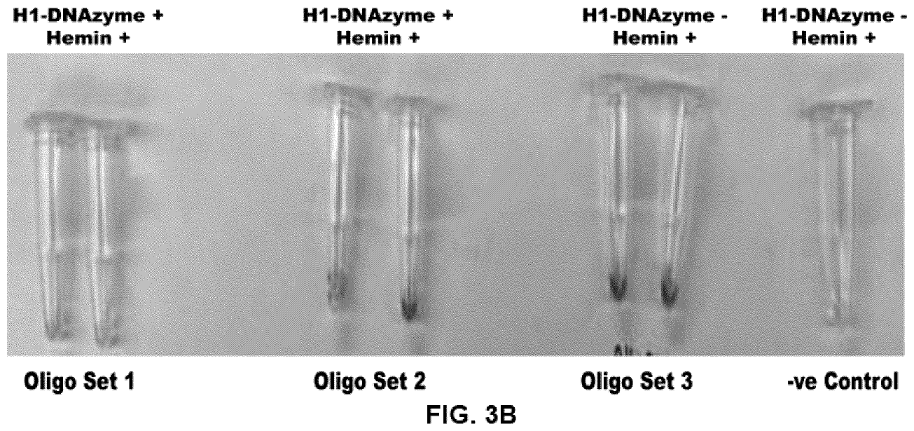

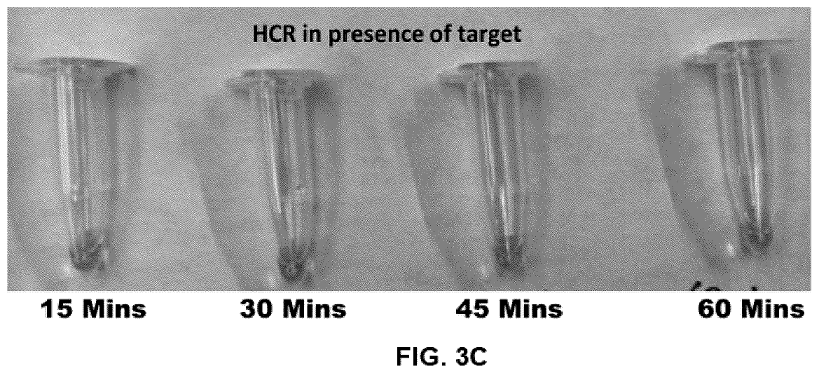

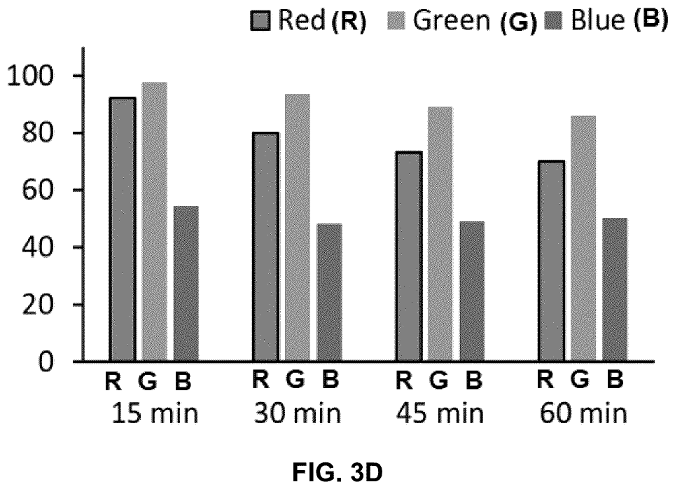

Colorimetry Optimization with H1-DNAzyme (Hairpin 1 DNAzyme). DNAzymes have been investigated to investigate their compatibility for colorimetric detection. A blue color was observed (FIG. 4A) upon quadruplex formation of DNAzymes after incubation with TMB and H₂O₂. The sustainability of the color was studied at two different time points (0 hours and 24 hours). After 24 hours the color was a greenish-yellow (FIG. 3A) and although different in appearance from the color exhibited at 0 hours this indicated successful entrapment of hemin into the G-quadruplex of H1-DNAzyme and its stability for an extended period of time. All three oligonucleotide hairpin DNAzyme pairs were investigated for their efficiency in generating color after incubation with TMB and H₂O₂. From the three hairpin DNAzyme sets, oligonucleotide set 3 showed the development of the highest color intensity after significantly less time compared to the other sets (FIG. 3B). Finally, 1 pg/μL of cardiac troponin I, was combined with H1-DNAzyme-set 3, H2-3, and aptamer-initiator. After HCR amplification the colorimetric output at different time points (FIG. 3C) was measured. The target-hairpin incubation time ranged from 15 minutes to 60 minutes to check the amplification performance in relation to time in the presence of the target. RGB analysis shows there is no significant change in the color intensity across this period indicating that 15 minutes of incubation time was sufficient for HCR to occur resulting in the generation of color, thus making the assay suitable for rapid detection of cardiac troponin.

Conclusion. A colorimetric assay was designed and tested and found to be rapid (15 minutes) and sensitive (fM), making it suitable for detecting cardiac troponin I at low levels. The universal approach deployed here for optimizing hairpin stability can be combined with more sensitive analytical techniques such as surface-enhanced Raman spectroscopy or hyperbranched chain amplification for improving the limit of detection. However, this method is complex, comparatively less specific, and needs multiple washing steps.

### Example 2: Femtomolar Detection of Cardiac Troponin I Using Surface-Enhanced Resonance Raman Spectroscopy (SERRS)

### Results

Principle of the assay. This work pioneered a universal method for detecting biomarkers. The initial step involved conjugating a biotinylated conformationally locked aptamer to streptavidin-MNP. This study used the notion of aptamer conformational change in the presence of a target. The aptamer employed in this work was developed to include an activator of downstream HCR amplification and a blocker sequence that is partially complementary to the aptamer's few nucleotides. These stable structures improve overall selectivity by allowing a variety of forces, including van der Waals, dipole-dipole, electrostatic, and others, to influence target identification. In the presence of the target cTnl, the magnetic nanoparticle-bound locked aptamer undergoes a conformational shift, and the unfolded initiator initiates the toehold-mediated hybridization chain reaction with structurally stabilized biotinylated hairpins (1 and 2). Streptavidin-coated gold nanostars with NIR-780 served as a transducing material for SERRS. The hypothesis was that the number of amplicons formed by the hybridization chain reaction would be exactly proportional to the amount of target, resulting in a stronger SERRS signal with more target (assay scheme shown in FIG. 4).

Screening of oligonucleotides by NUPACK software and validation of cross identity by nucleotide BLAST. To provide proof of concept, hairpins and oligonucleotides were screened in NUPACK software, which is required to identify any cross reaction. The initiator sequence was chosen from a plant species to prevent cross-hybridization with host or human species. The sequence similarity of the initiator sequence was examined using nucleotide BLAST, which revealed no resemblance to the human genome sequence.

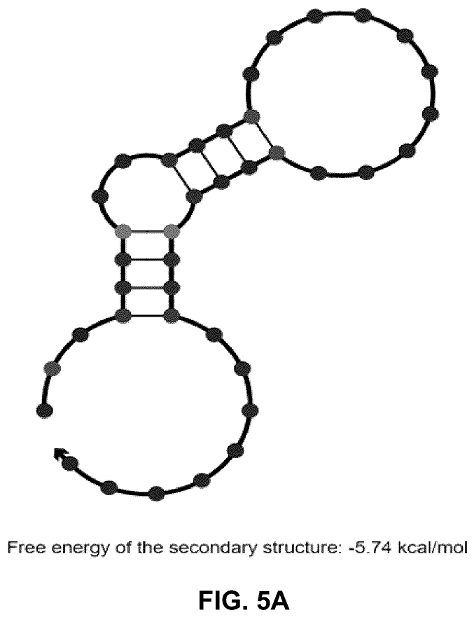

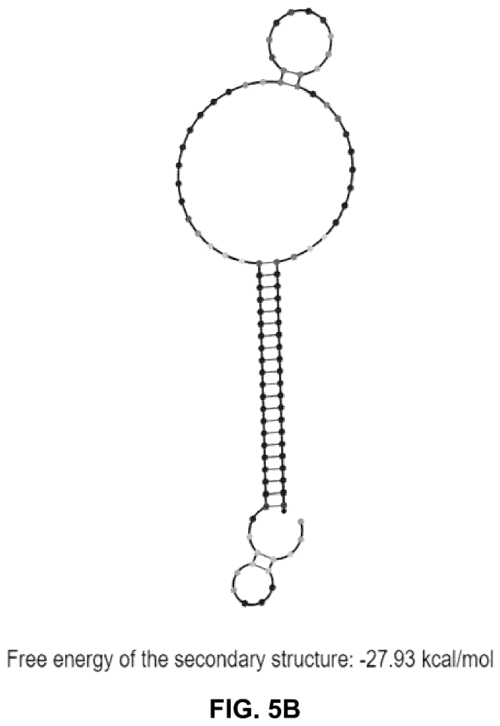

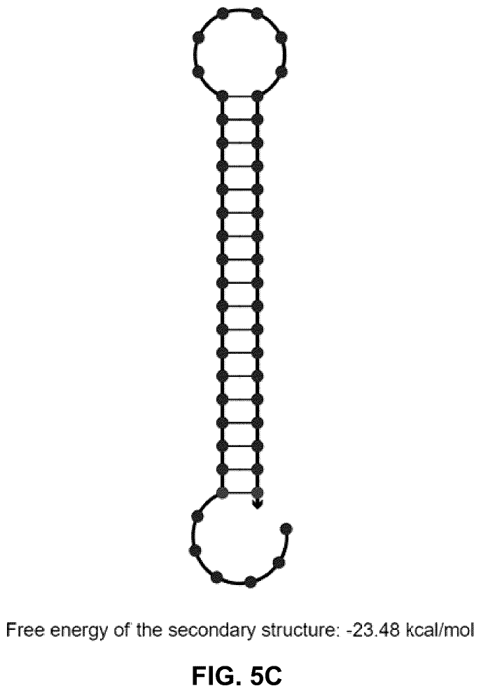

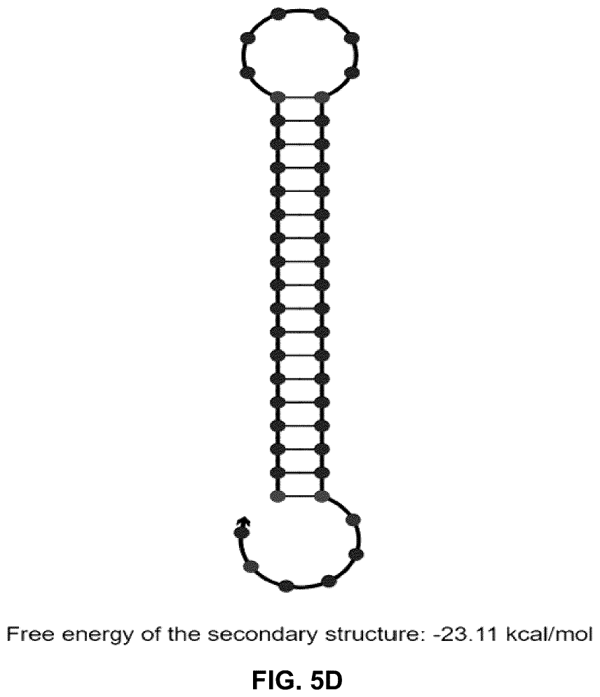

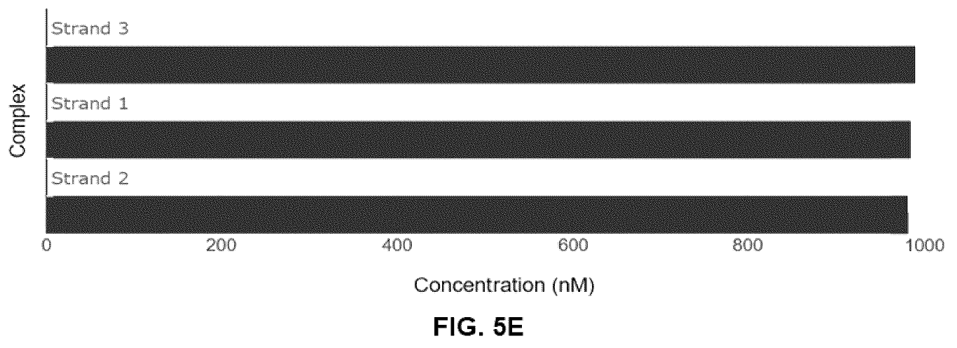

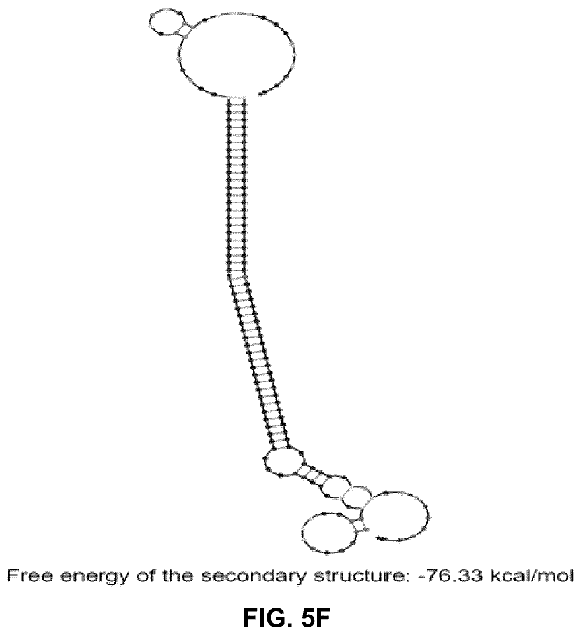

The aptamer's 3′ end contains the initiator of the downstream hybridization chain reaction. To make it a closed or locked configuration, the blocker sequence (complementary to the few nucleotides of the aptamer) was appended to the 3′ end of the initiator. The locked configuration was employed to improve the selective opening of the aptamer in the presence of a target. The free energy of a locked aptamer shape (FIG. 5B) was found to be significantly lower than that of the actual aptamer (FIG. 5A), indicating greater stability. Following confirmation of the secondary structure, the designed aptamer in closed configuration, hairpin 1 (FIG. 5C) and hairpin 2 (FIG. 5D) were simulated in NUPACK to observe complex development. The simulation uses a concentration of 1 μM for each oligonucleotide and a temperature of 37° C. It was observed that no product is formed while the aptamer is in closed confirmation (FIG. 5E). After that, hairpins were simulated with the initiator integrated blocker sequences (because, in the presence of a target, the engineered aptamer should be unavailable for hairpin binding), and chain formation (FIG. 5F) was observed, which confirmed the hypothesis about oligonucleotide opening and specific binding. It was also discovered that the aptamer's blocker sequence interferes with the opening of hairpin 2, resulting in some sections of hairpin 2 remaining unused in the assay. However, the amplicons are still in large quantities that may be easily integrated with any readout approaches, and the main approach of the assay is connected with specificity, thus the structures and concentrations utilized in the simulation were carried over to subsequent studies.

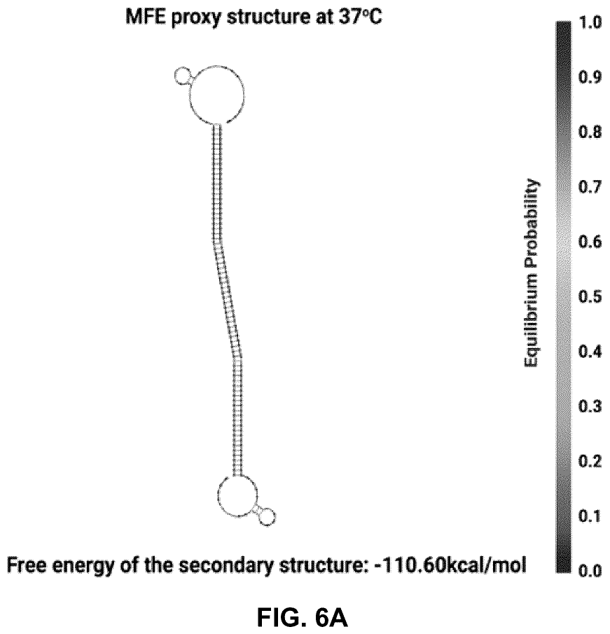

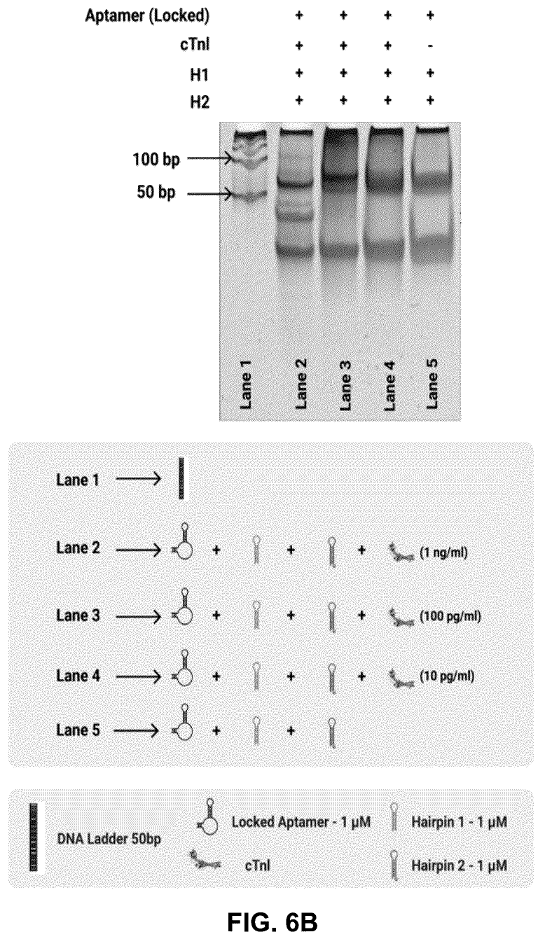

Validation of NUPACK simulation of oligonucleotides by gel electrophoresis and imaging study. To look at the simulation of the amplicon from NUPACK, gel electrophoresis analysis was done. The amplicon formation was checked with 15% Native PAGE in the presence of varying target concentrations. Before adding hairpins 1 and 2, the snap-cooled locked aptamer was incubated for an hour at various target concentrations (1 ng/mL, 100 pg/mL and 10 pg/mL). There were several bands formed in the gel at the higher target concentration of 1 ng/mL. This finding suggests that there are many aptamer openings linked to the MNP in the presence of high target concentration, which results in several short amplicons (FIG. 6B, Lane 2). Similarly, for the lower concentration, as the hairpin concentration was the same for all reactions, a smear-like pattern was seen in the gel close to the lane, indicating a high length chain formation in the presence of a lower quantity of target (FIG. 6B, lane 3 and lane 4). The gel holes prevented the high molecular weight amplicon from migrating with ease. Since the initiator section was not opened in the absence of the target cTnl, which indicates the absence of amplicons in the reaction as well as in the gel electrophoresis investigation, no multiple band development was observed for the negative control (FIG. 6B, lane 5).

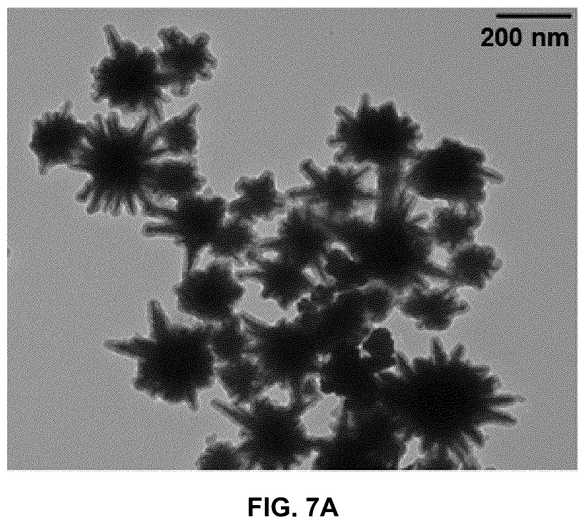

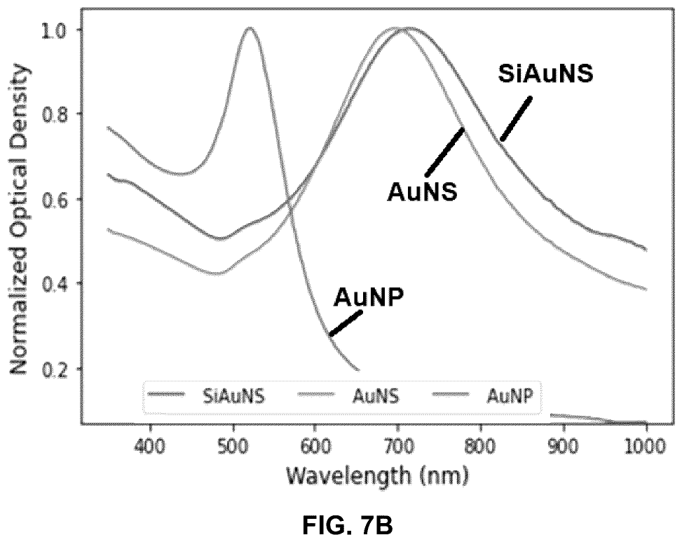

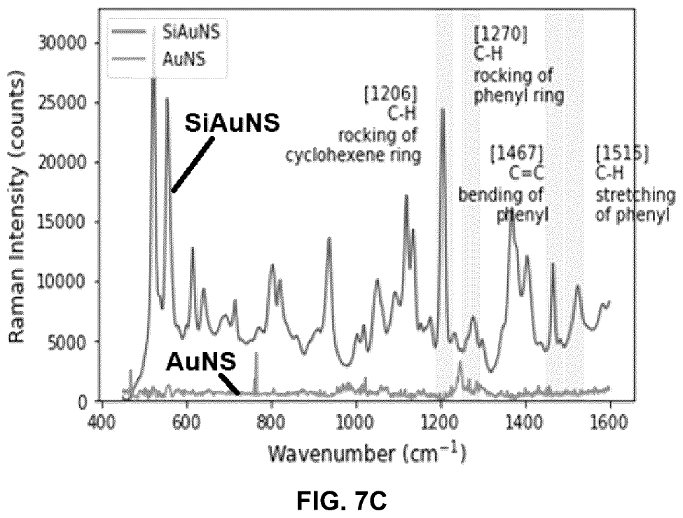

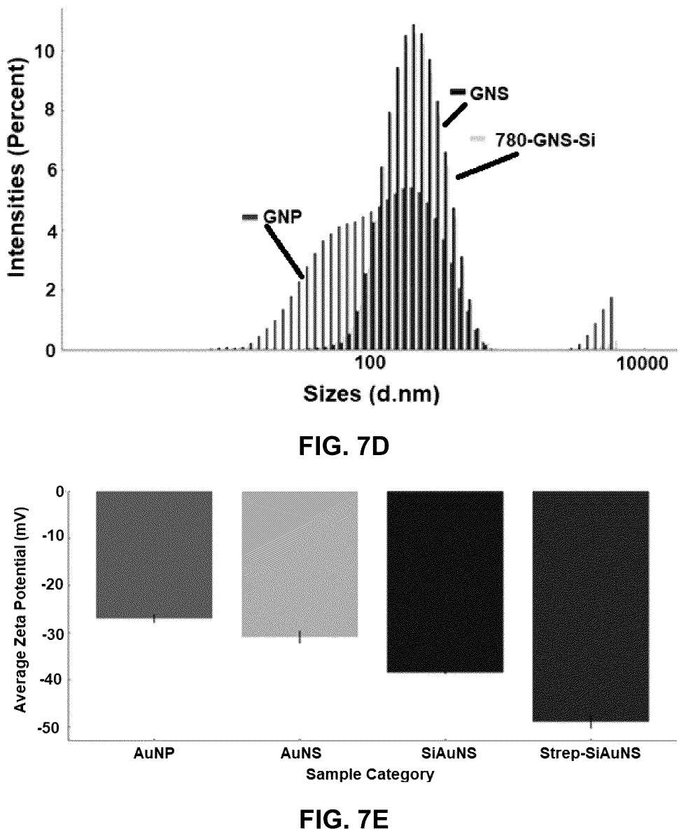

Characterization of gold nanoparticles. The Transmission Electron Microscopy (TEM) images (FIG. 7A) provide a definitive visualization of the gold nano-star particles, which are depicted with a high degree of structural clarity. The nano-stars exhibit a morphology consistent with the desired star-shaped geometry, and a thin, uniform layer of silica coating is clearly discernible, enveloping the surface of each gold nano-star. This silica layer is integral to the functionality of the nano-stars, as it contributes to the colloidal stability and biocompatibility of the particles. The uniformity of the silica coating, as evidenced by the TEM results, suggests a successful synthesis. Moreover, the complete coverage of the gold nano-stars by the silica layer is essential for subsequent reporter molecule functionalization. It ensures that the intrinsic properties of the gold core are preserved while providing a versatile platform for further surface modifications.

The UV-Vis spectroscopy analysis (FIG. 7B) further confirms the successful synthesis and modification of the gold nano-stars. The spectral data reveal a rightward shift in the peak wavelength following the addition of the silica coating, as indicated by the comparison between the AuNS and SiAuNS spectra. This redshift is consistent with the expected optical behavior when a dielectric material, such as silica, is deposited onto the surface of plasmonic nanoparticles. The silica shell increases the effective particle size and alters the local refractive index around the nano-stars, which, in turn, influences their plasmonic resonance wavelength. The observed shift in the absorbance peak thus serves as a spectral signature, indicative of the presence of the silica coating on the nano-star surface. This shift corroborates the TEM findings, which visually confirmed the coating. It provides a quantifiable measure of the modification, reinforcing the evidence for successfully encapsulating the gold nano-stars with a silica layer. The SERRS measurements (FIG. 7C) demonstrate the enhanced Raman signals obtained from the silica-coated gold nano-stars compared to the bare gold nano-stars, which is attributed to the localized surface plasmon resonances of the gold nano-stars that significantly amplify the Raman scattering of molecules close to their surface. The data reveal four characteristic peaks: a peak around 1206 cm⁻¹ indicates C—H rocking of cyclohexane ring, another peak at approximately 1249 cm⁻¹ indicative of bending modes in aromatic compounds, peak at 1337 cm⁻¹ corresponding to the C—N bond stretching vibrations, a peak around 1573 cm⁻¹ suggesting C═C stretching vibrations in aromatic rings, and a peak near 1610 cm⁻¹ associated with C—C stretching vibrations in aromatic structures. The presence and intensity of these peaks confirm the successful attachment of specific functional groups to the nano-stars and demonstrate the SERRS technique's high sensitivity. Significantly, the silica coating does not diminish the SERRS effect, ensuring the method's utility for sensitive detection and spectroscopic analysis in various applications. This enhancement is critical for the intended applications, as it provides a robust platform for the sensitive detection and detailed characterization of molecular species. The Dynamic Light Scattering (DLS) data, confirm the achievement of a uniform and well-controlled synthetic process, culminating in nanoparticles with potential for the present application (FIG. 7D). The DLS results also reflect a monodisperse size distribution for the nanoparticles, with a PDI value of approximately 0.222. This value is within the acceptable range for colloidal solutions, suggesting that the nanoparticles are relatively uniform in size with minimal aggregation. The sharp peak in the intensity distribution graph further corroborates the uniformity in the size of the synthesized nanoparticles.

The observed downward trend in the zeta potential from the gold nano-sphere seeds to the silica-coated, dye-encapsulated gold nano-stars substantiates the successful modification of the nanoparticles' surfaces. Specifically, the further decrease in zeta potential for the SiAuNS sample compared to the uncoated AuNS confirms the presence of the silica coating. The silica shell, which inherently possesses a dense layer of negatively charged silanol (Si—OH) groups, contributes to the increased negative charge on the nanoparticle surface. This modification not only enhances the colloidal stability of the nano-stars but also directly indicates the successful coating process. Further decrease in zeta potential for the sample streptavidin-SiAuNS compared to the SiAuNS confirms the successful coating of streptavidin (FIG. 7E). The magnitude of the zeta potential change serves as a proxy for the completeness and uniformity of the streptavidin coating, which is pivotal for the particle's performance in the intended application.

The results imply that the synthetic process is well-controlled, yielding nanoparticles likely to exhibit predictable behavior during intended use. This data supports the successful synthesis of nanoparticles with the potential for high stability and uniformity.

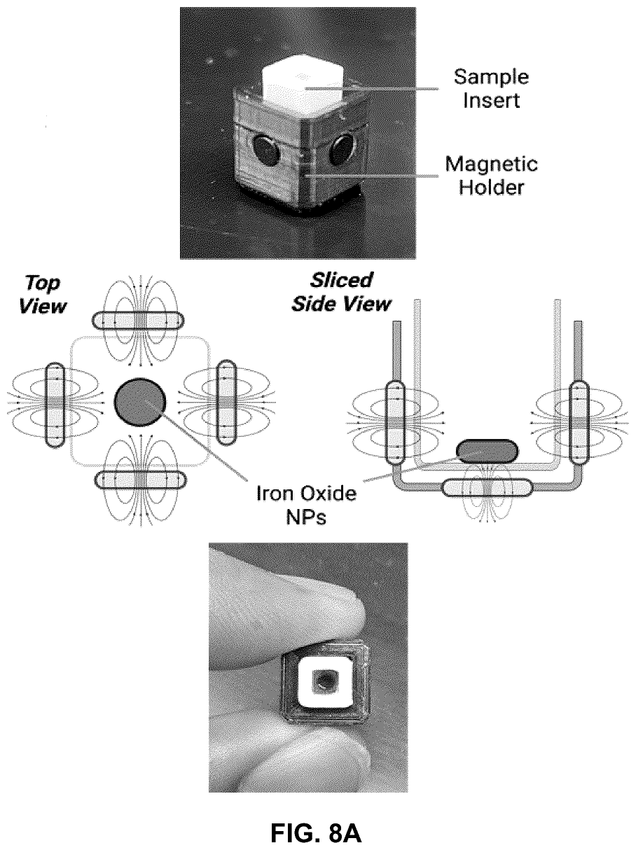

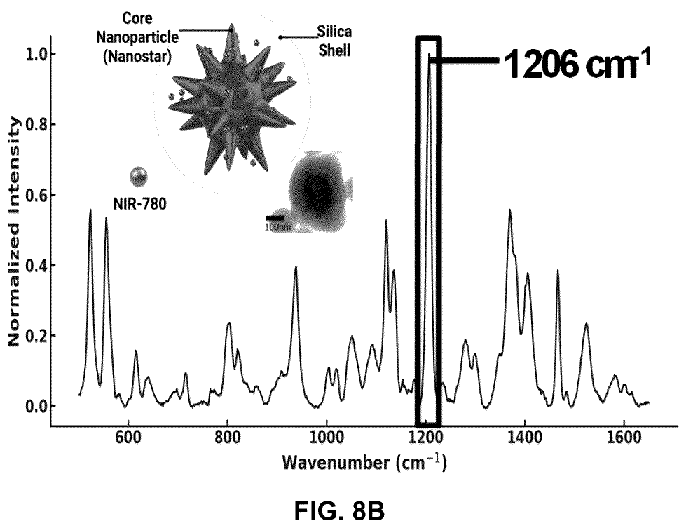

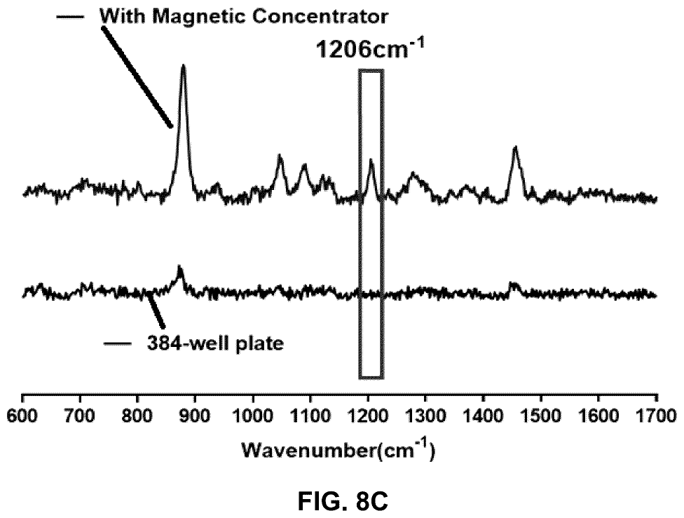

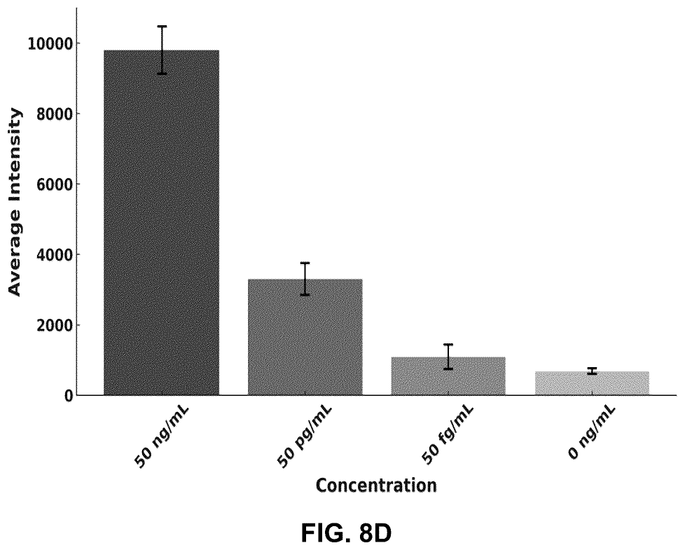

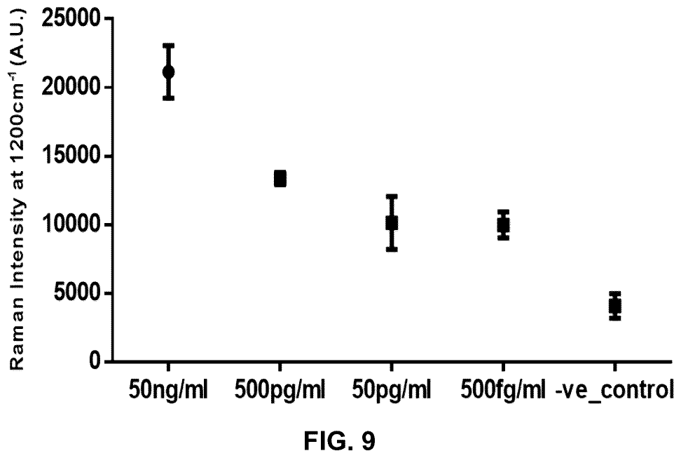

Determination of the LoD of the assay using SERRS. As described in FIG. 4, a quantitative strategy was used with magnetic beads coupled with locked aptamer-initiator, hybridization chain reaction (HCR) and streptavidin coated gold nanostars (Si—AuNS) to achieve a proof-of-concept for cardiac troponin-I (cTnl) detection at the point of care. Gold nanostars covered with silica served as the SERRS substrate (reporter dye), while NIR-780 dye was used as the Raman reporter. A biotin-streptavidin linkage was used to immobilize aptamer sequences unique to cTnl on streptavidin-coated MNP. With the aptamer confirmation in a “locked beacon state,” it would only open selectively when the cTnl target was present. Hairpins 1 and 2, initially sequestered in a stem-loop confirmation, became available to bind to the cTnl-aptamer complex through toehold binding. Hybridization chain reaction (HCR) ensued, marked by successive stacking of the hairpins. Hairpin 2, possessing a biotinylated end, was then available to bind to streptavidin-SiAuNS. Subsequent washes removed unbound Si—AuNS particles, rendering the HCR assemblies of dynabeads-Si—AuNS prepared for measurement. First, the magnetic nanoparticles integrated streptavidin coated gold nanostar coupled HCR assemblies were checked in 384 well plate. Very low or no Raman characteristic peak intensities (FIG. 8C) were observed due to uneven distribution of magnetic nanoparticles in 384 well plate. To address this issue a 3D printed magnetic holder was designed to pull down all magnetic nanoparticles at single point to increase the Raman intensity. The 3D-printed sample insert, designed to fit into a holder containing five 2 mm N52 magnets (four on each side and one at the bottom, described in FIG. 8A).

Two sets of experiments were conducted. Set 1 featured aqueous solutions of cTnl at concentrations of 50 ng/mL, 50 pg/mL, and 50 fg/mL. 1 μM of hairpins and 5 μL of streptavidin gold nanostar (OD 0.6) was introduced in the assay. The assay was employed with a gold nanostructure with NIR 780 reporter dye since it is resonant with the 785 nm Raman spectrophotometer for final readout quantification. The nanostar shape of the nanoparticles can generate more hotspots which can improve the assay's sensitivity. FIG. 8B depicts the Raman spectra of a gold nanostar encapsulated with NIR780, with the peak at 1206 cm⁻¹ (Vibrational mode for NIR780's C—H cyclohexane ring) being particularly conspicuous. FIG. 8D shows a concentration-dependent rise in SERRS intensity at 1206 cm⁻¹ for all the concentrations tested. The broad cTnl concentration range allows for potential discrimination between normal patients and those with early onset cardiac injury, aligning with the American Heart Association's (AHA) established levels (>40 pg/mL) indicative of a recent cardiac event.

For set 2, an analysis was conducted of the dynamic range of cTnl in all cases of cardiac damage, including heart failure and recent heart attacks. Concentrations of 500 fg/mL, 50 pg/mL, 500 pg/mL, and 50 ng/mL of cTnl were added to cTnl-free serum that had been subjected to an optimal 56° C. treatment period to inactivate IgG. As previously mentioned, the presence of serum components like albumin, which can overlap with the Raman spectrum of the reporter molecule, was responsible for the greater intensity of Raman peaks seen. Furthermore, a distinct interference of serum is seen in the peak intensities for lower values.

Higher concentrations of hairpins (2 μM) and higher amount of (15 μL) of streptavidin-SiAuNS (OD-0.6) were added to the test in order to address this problem and optimize the SERRS signal. Assay-based SERRS measurements were performed on serum containing 0.5, 10, 25, 50, and 100 pg/mL of cTnl in order to examine the sensitivity at lower concentrations. Raman spectrograms show a progressive rise in peak intensity at 1206 cm−1 when cTnl concentrations rise between 0.5 and 100 pg/mL (FIG. 10A). There was a strong linear correlation found between the cTnl concentration and Raman intensity. The estimated detection limit, based on the IUPAC 3σ/m criteria, is 0.5 pg/mL (FIG. 10B). The suggested SERRS technique exhibits ease of use, quick reaction times, and a broad detection range by utilizing HCR assemblies coupled with streptavidin SiAuNS. This method serves as a compelling proof-of-concept for the detection of cTnl from patient serum samples at the point of care.

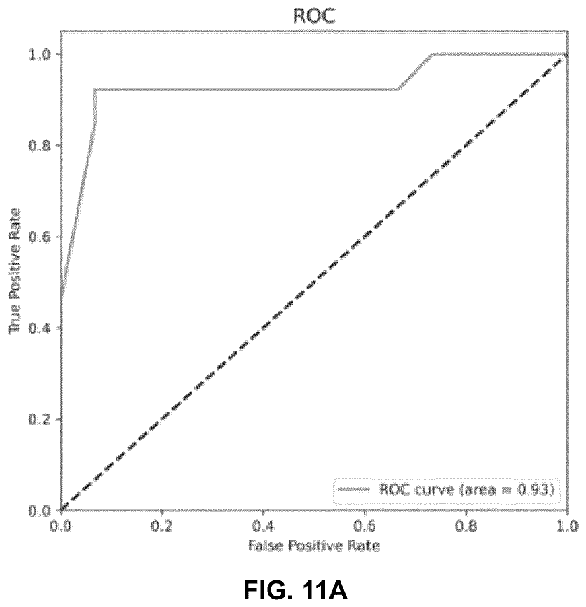

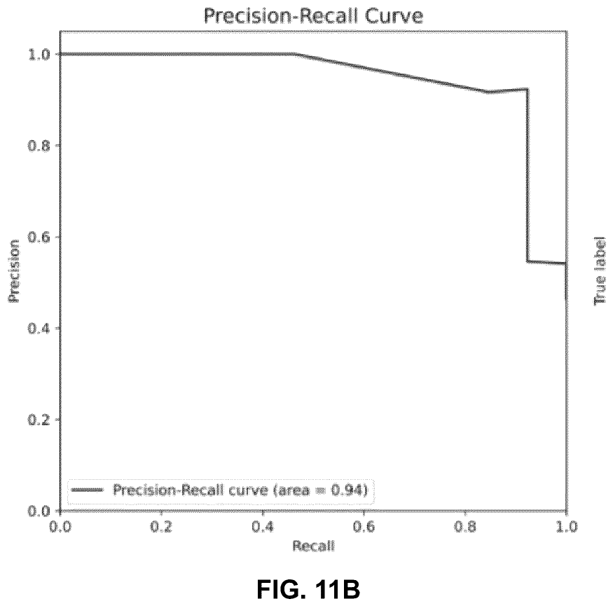

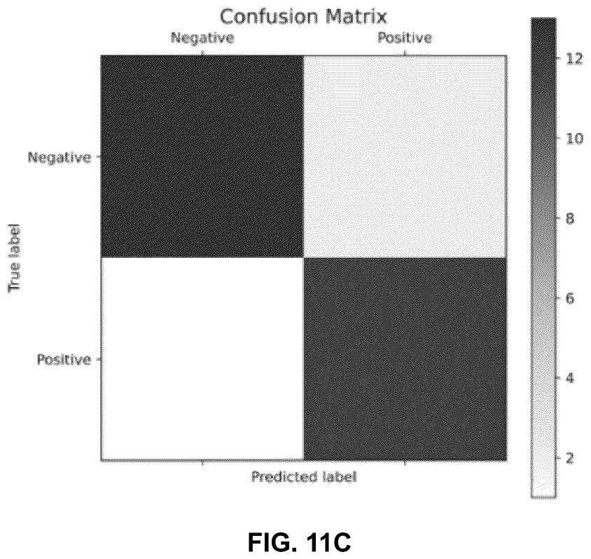

Real sample analysis. To test the assay's effectiveness, clinical serum samples (N=37, supplied by the UCLA Department of Bioengineering and stored at −20° C.) were obtained and cTnl value were measured by standard ELISA method. Following that, the samples were subjected to the assay and the Raman spectra was collected. The primary objective was to categorize the samples based on their spectral data, distinguishing whether cTnl levels were above or below the clinical threshold of 40 pg/mL. The dataset was analyzed employing nine distinct statistical models, and the efficacy of these models was appraised through metrics such as accuracy, precision, recall, F1 scores, and ROC AUC, as depicted in FIGS. 11A-11C. Among the evaluated models, the Random Forest classifier emerged as the most effective, achieving scores of 0.89 for accuracy, 0.86 for precision, 0.92 for recall, 0.89 for the F1 score, and 0.93 for the ROC AUC. FIGS. 11A-11C illustrate the ROC curve, the precision-recall curve, and the confusion matrix, providing a visual representation of this model's performance.

### Materials for SERRS Experiment

Materials. The cTnl used in these experiments was acquired from Genescript, Piscataway, New Jersey, USA. All the oligo nucleotides were acquired from Integrated DNA Technologies (Iowa, USA). All chemicals were purchased from Millipore Sigma. cTnl-free human serum was purchased from HyTest (Finland). Streptavidin was ordered from Sigma (US). The ELISA kit for cTnl was purchased from abcam (UK). The patient's samples were obtained from UCLA, Department of Bioengineering.

Design of the locked aptamer and NUPACK simulations. The initiator sequence has been chosen from the literature and cross verified for its absence in humans, or pathogens by nucleotide BLAST. Then the blocker sequence which is complementary to the few nucleotides of aptamer was added to the 3′ end of the initiator to conformationally close its structure. The initiator and blocker sequence was added to the 3′ end of the aptamer to make it a locked aptamer. The presence of minimal secondary structure of the initiator sequence and blocker sequence at its 3′ end of aptamer was simulated in NUPACK and RNAfold to predict its secondary structure. If the initiator sequence with blocker sequence is found to form a secondary structure with the aptamer, then the initiator sequence has been shuffled to obtain a fresh initiator to avoid the unwanted secondary structures. The hairpin 1 has been designed on NUPACK on partial complementarity and introducing loop with random repeat sequence. The hairpin 2 was designed on the basis of the partial complementarity with the H1. Finally, the complex formulation simulation was checked in NUPACK. All the oligonucleotide sequences were shown in Table 1.

TABLE 1
Sequences Used

| | | | |
|---|---|---|---|
| | | | SEQ |
| Description | Sequence (5′→3′) | Notes | ID NO. |

Sequences used in Colorimetry Studies

| | | | |
|---|---|---|---|
| Aptamer | TTTTTCGTGCAGTACGCAACCTTT | Italics: Aptamer Tro4 | 1 |
| Sequence | CTCATGCGCTGCCCCTCTTAAAAA | Underlined: Spacer | |
| | AAAGCAGATCCTAAGCCGCACCC | Bold: Initiator | |
| | AATTAAGGGGCAGCGCATGAGAA | Underlined and italics: | |
| | AGGTTCCGTA | Blocker | |
| Initiator | GCAGATCCTAAGCCGCACCCAAT | | 2 |
| Sequence | T | | |
| HTG-H1 | AATTGGGTGCGGCTTAGGATCTG | Underline: Multimeric G- | 3 |
| | CACAAAGGCAGATCCTAAGCCGC | Triplex/Quadruplex Sequence | |
| | ACTGGGAAGGGAGGGTGGGAAG | | |
| | GGAGGGTGGGAAGGGAGGG | | |
| H1 | GCAGATCCTAAGCCGCACCCAAT | Italic: Initiator of hyper- | 4 |
| | TGTGCGGCTTAGGATCTGCCTTT | branched chain reaction | |
| | GTAGAAGAAGGTGTTTAAGTA | | |
| HTG-H2 | AGGGCGGGTGGGTGTTTAAGTTG | Underline: Multimeric G- | 5 |
| | GAGAATTGTACTTAAACACCTTCT | Triplex/Quadruplex Sequence | |
| | TCTTGGGT | | |
| H2 | TGGGTCAATTCTCCAACTTAAACT | | 6 |
| | AGAAGAAGGTGTTTAAG | | |

Sequences Used for SERRS Studies

| | | | SEQ |
|---|---|---|---|
| Description | Sequence (5′→3′) | Notes | ID NO. |
| Aptamer | TTTTTCGTGCAGTACGCAACCTTT | Italics: Aptamer Tro4 | 1 |
| Sequence | CTCATGCGCTGCCCCTCTTAAAAA | Underlined: Spacer | |
| | AAAGCAGATCCTAAGCCGCACCC | Bold: Initiator | |
| | AATTAAGGGGCAGCGCATGAGAA | Underlined and italics: | |
| | AGGTTCCGTA | Blocker | |
| H1 | AATTGGGTGCGGCTTAGGATCTG | | 7 |
| (Biotinylated) | CACAAAGGCAGATCCTAAGCCGC | | |
| | AC | | |
| H2 | GCAGATCCTAAGCCGCACCCAAT | | 8 |
| | TGTGCGGCTTAGGATCTGCCTTT | | |
| | GT | | |

Oligonucleotide screening. The aptamer (1 μM) was incubated with different concentration of (1 ng/mL, 100 pg/mL, 10 pg/mL) cTnl for 1 hour. After, 1 μM of biotinylated hairpin 1 (H1) and biotinylated hairpin 2 (H2) was then added and incubated for a further 45 minutes. Water was used in place of cTnl for the control samples. All reactions were mixed with 6× gel dye and loaded into the 15% native polyacrylamide gel (PAGE) vertical gel system.

Native PAGE analysis. The native PAGE gel was made by adding 6 mL of 30% acrylamide and bisacrylamide solution (29:1), 6 mL of 1× Tris-boric acid -EDTA buffer (TBE buffer), 100 μL of 10% (w/v) ammonium persulphate (APS) and 20 μL TEMED. The solution was transferred to a vertical gel system and allowed for 30-45 minutes to solidify. The combs were removed and the aptamer, hairpin, and cTnl combined reactions mentioned previously were loaded into the gel.

Synthesis of gold nanostars. A solution with a concentration of 0.25 mM gold chloride (HAuCl₄) is formulated by diluting 1 M HCl at a 1000:1 ratio. Following this, 2 mL each of 25 mM ascorbic acid and 0.5 mM AgNO₃ are concurrently introduced into 50 mL of the aforementioned solution, with continuous stirring. This leads to the immediate formation of gold nano-star particles, evidenced by the solution's dark blue hue. The mixture is stirred vigorously for 30 seconds to ensure uniformity. After mixing, the solution is divided into two 50 mL centrifuge tubes and centrifuged at 3000 rcf for 30 minutes. The supernatant is then discarded, and the pellet is resuspended in 25 mL of DI water. Finally, the resuspended solution is filtered using a 0.2 micron nitrocellulose membrane.

Synthesis of silica coating embedded with Raman reporter dye (NIR-780) followed by streptavidin coating. The reaction is initiated by manually adding 36 mL of ethanol (EtOH) to the flask. This is followed by the addition of 3 mL of an aqueous ammonia (NH₃ in H₂O) solution, and the mixture is stirred for 5 minutes. Afterward, 3.6 mL of gold nano-star particles (OD˜0.6) are added, and the mixture is stirred for an additional 5 minutes. Separately, Solution X is prepared by combining 90 mL of isopropanol (IPA), 1.2 mL of tetraethyl orthosilicate (TEOS), 5.4 mL of a 1×10⁻⁶ M NIR-780 solution, and 9 mL of distilled water (DI H₂O). This solution is then added to the flask containing the initial mixture, and the entire solution is mixed at a rotation speed of 700 rpm for 20 minutes. The mixture is then divided evenly into six 50 mL centrifuge tubes, and ethanol is added to each tube to bring the total volume to 50 mL. These tubes are then centrifuged at 20,000 relative centrifugal force (rcf) for 20 minutes, forming dark blue pellets at the bottom. The supernatant is removed from each tube, and the pellets are mixed with 2 mL of ethanol. The mixture is then transferred to 2 mL tubes for washing, repeated four times with ethanol and at least two times with DI water to remove any excess. Finally, 2 mL of DI water is added to resuspended the solution, which is then ready for further surface modification.

The methodology for streptavidin functionalization of SiAuNS was adapted from the literature. To functionalize streptavidin on Si—AuNS, 500 μL of Si—AuNS (OD˜0.6) was aliquoted in 10% ammonium hydroxide with 1.5 μL of 30% 3-Aminopropyltriethoxysilane. A further 3 hours of incubation on the rotating shaker at room temperature would be necessary, followed by two 10-minute washing processes with DI water at 5000 rpm. After resuspension in water, 10 μL of 10 mM N-ethyl-N′-(3-(dimethylamino) propyl) carbodiimide, 10 μL of 25 mM N-hydroxysuccinimide, and 5 μL of 1 mg/mL streptavidin were added to the pretreated SiAuNS and incubated overnight at 4° C. The functionalized nanoparticles were washed twice with DI water at 5000 rpm for 10 minutes, then resuspended in PBS.

Characterization of streptavidin coated silica-gold nanostar. The optical stability of silica-coated, dye-encapsulated seedless SiAuNS and bare AuNS particles was characterized using a Tecan Infinite 200 Pro microplate reader, revealing strong extinctions at 760 nm and 750 nm, respectively. Transmission electron microscopy (TEM) images were acquired using a JEOL 1200, and size analysis was performed using ImageJ. To obtain TEM images, 8 μL of the 10-fold diluted, silica-coated, dye-encapsulated SiAuNS and AuNS particles were deposited onto a carbon film-coated copper grid and left to dry at room temperature for 24 hours. Prior to introduction into the well plate, the sample undergoes sonication to ensure homogeneity. A volume of 100 μL of the prepared sample solution is then meticulously transferred into the designated wells of a black well plate. The laser system, configured with an excitation wavelength of 785 nm, is optimized to emit at a power level of 31.2 W, with the laser nozzle positioned at a distance of 4.5 cm from the surface of the well plate. For the Si AuNS particles, an integration time of 0.5 s is applied consistently. The concentration of the samples introduced into the well plate is consistently maintained at an optical density (OD) of approximately 0.6. A solution of gold nano-sphere seed, pre- and post-silica-coated, dye-encapsulated gold nano-stars (optical density ˜0.6) was subjected to sonication to mitigate aggregation. To prepare for analysis, a dilution was performed by combining 100 μL of the initial solution with 900 μL of deionized water. The resulting mixture was then sonicated to ensure homogeneity. Particle size distribution and zeta potential were assessed using a Malvern Zetasizer Nano ZS. A volume of 1 mL from the diluted sample was employed for Dynamic Light Scattering (DLS) measurements. Subsequently, a portion of 800 μL from this dilution was utilized to determine the zeta potential.

Assay with the magnetic nanoparticles and cTnl in aqueous solution and in serum. 10 μg of streptavidin coated magnetic nanoparticles (MNP) (Invitrogen) were washed with washing buffer (25 mM Tris, 150 mM NaCl, pH 7.2, 0.1% BSA, and 0.05% Tween-20 detergent). Subsequently, the snap cooled (heated at 95° C. for 7 minutes followed by incubation in ice) biotinylated aptamer was dissolved in 1× binding buffer (Tris 10 mM, 1 mM EDTA, 1000 mM NaCl) and incubated with the washed MNPs overnight whilst undergoing gentle mixing. The aptamer-MNP complex then carefully washed with 1× binding buffer and the complex were blocked with 1% BSA solution for 45 minutes. After washing the complex was dissolved with high salt concentration based binding buffer (20 mM Tris (pH 7.6), 1 mM EDTA, 1000 mM NaCl, 5 mM KCl, 2 mM MgCl₂, 1 mM CaCl₂) with added sodium phosphate. Following that the desired concentration of cTnl in PBS buffer was added to the solution. After 60 minutes of incubation hairpin 1, hairpin 2 (final conc. 1 μM) and 5 μL of streptavidin coated SiAuNS (OD-0.6) was added to the reaction mixture and again incubated for 60 minutes. The whole complex was washed with sodium phosphate and sodium chloride buffer. The resultant MNP-cardiac troponin —HCR chain-Au Nanostar complex was dissolved in water and then Raman analysis was done. For serum spiked sample at first the serum was heat inactivated at 56° C. for ten minutes and aliquoted in small tubes. After that desired concentrations of cTnl was spiked into the heat inactivated serum. The rest of the steps were exactly followed as described in the previous section.

Assay with the clinical samples. The aptamer MNP complex was made as described before. The aptamer-MNP complex then carefully washed with 1× binding buffer and the complex were blocked with 1% BSA solution. After washing the complex was aliquoted and dissolved with high salt concentration based binding buffer with added sodium phosphate. After washing 25 μL of different clinical samples (N=37) obtained from patients was added to the respective MNP aliquot solution. After 60 minutes of incubation hairpin 1 and hairpin 2 was added to the reaction mixture with a final concentration of 2 μM and 15 μL of streptavidin coated SiAuNS (OD-0.6). The complex was again incubated for 60 minutes. The whole complex was washed with sodium phosphate and sodium chloride buffer. The resultant MNP-cardiac troponin —HCR chain-Au Nanostar complex was dissolved in water and then Raman analysis was done. The whole reaction was done in the BSL-2 containment.

Raman spectra analysis. To facilitate SERRS measurements, a custom 3D-printed sample holder compatible with the Wasatch Photonics Raman 785p setup was employed. Comprising two 3D-printed units—a cuboidal unit with an open face and embedded magnets as the base, and a male container serving as the sample holder with a maximum volume of 30 μL—the setup was calibrated using Raman spectra of ethanol to determine the optimal focal length. SERRS analysis is carried out using a Wasatch Photonics 785p benchtop Raman spectrometer (Wasatch Photonics, Utah, USA). The benchtop device operates at 785 nm (31.2 mW) and is equipped with a point and shoot adjustable tip. The distance from the device aperture to tip end is set at ˜20 mm from the edge of the secondary male container, this was found to be the optimal distance for SERRS interrogation of the HCR assemblies of dynabeads-Si—AuNS. 3 second integration time was used for buffer spiked cTnl study. Additionally a 10 second integration time was used for serum spiked cardiac troponin I and clinical sample study. The raw spectra were smoothened and baseline corrected using the asymmetric least squares algorithm on MATLAB and were processed for downstream analysis.

Clinical sample classification by deep learning based on cTnl levels. Samples were procured from 37 individuals, subsequently classifying these based on their cTnl concentrations, as determined via ELISA, into two categories: low (<40 pg/mL, n=15) and high (>40 pg/mL, n=22). To enhance the reliability of the spectral measurements and mitigate variability, each sample was analyzed three times, with the mean of these readings serving as the basis for further analysis. Preprocessing of spectral data started with truncation, retaining only the spectral peaks within the 400-1600 cm−1 range. Baseline correction was subsequently performed using the asymmetric least squares method, with smoothness and asymmetry parameters set at 1×10⁶ and 1×10⁻², respectively.

To address the issue of cosmic rays, smoothing was applied through the Savitzky-Golay method, opting for a window size of 9 and employing cubic polynomial fitting. To reduce the dataset's complexity, peak picking was used to distill the spectral data into 36 discernible features. Prior to feature scaling, a logarithmic transformation was applied to the data, to normalize the distribution and mitigate the effects of skewness. Feature scaling was then conducted to standardize the dataset, ensuring that no single feature disproportionately influenced the model due to its scale. Given the observed class imbalance among these samples (15 low and 22 high), undersampling via one-sided selection was employed. This approach was specifically chosen to counteract the potential bias towards the majority class, thereby fostering a more balanced and equitable model training environment. The analytical framework encompassed the evaluation of nine distinct statistical models, including the Support Vector Classifier, Random Forest, Logistic Regression, K-Neighbors Classifier, Decision Tree, Gradient Boosting, AdaBoost, Gaussian Naive Bayes, and XGBoost. To identify the optimal hyperparameters for each model, a Gridsearch coupled with leave-one-out cross-validation (LOOCV) was employed. The performance of each optimized model was rigorously assessed using LOOCV, focusing on key metrics such as accuracy, precision, recall, F1 score, and ROC AUC (where applicable).

Assay Compatibility with Colorimetry

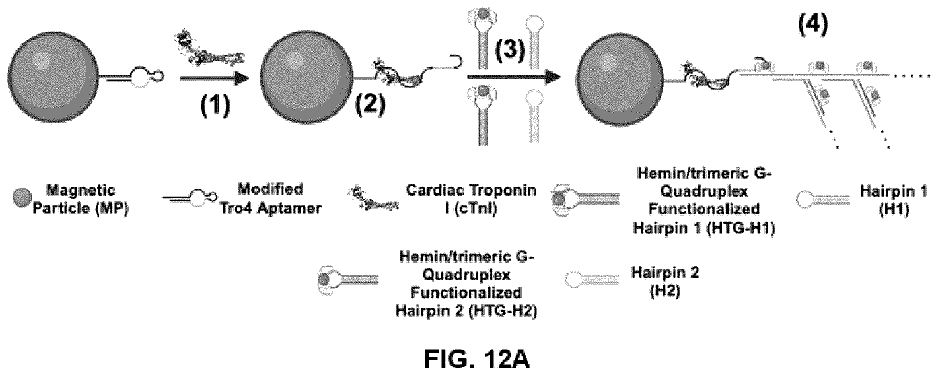

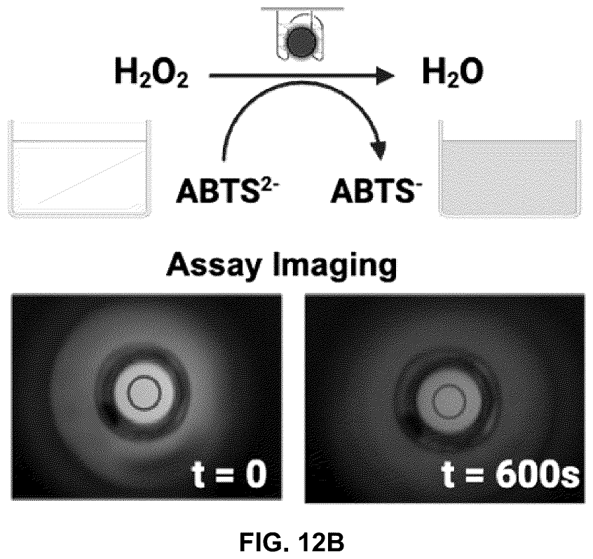

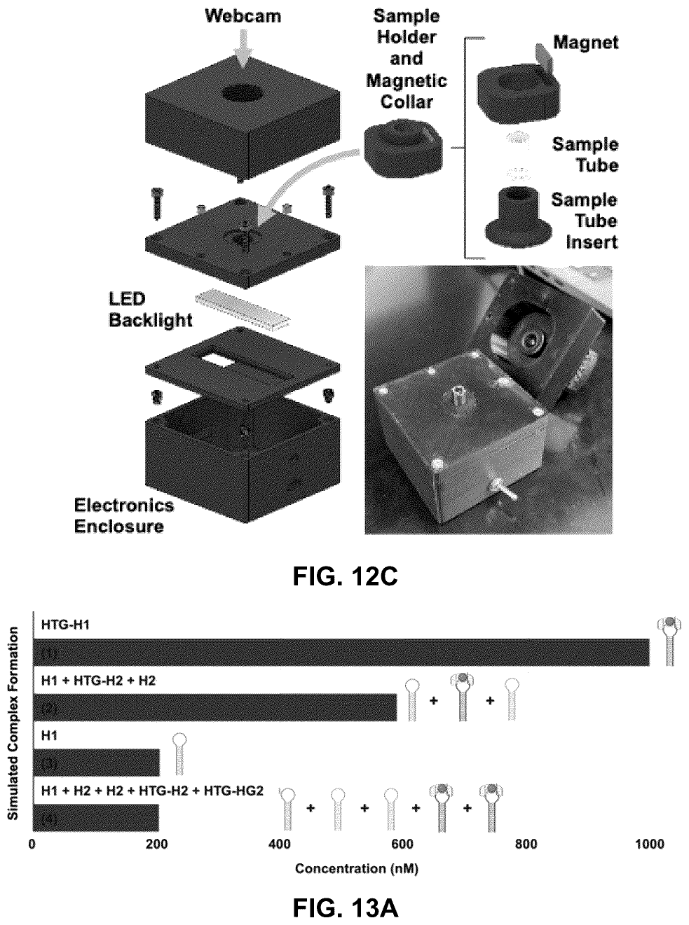

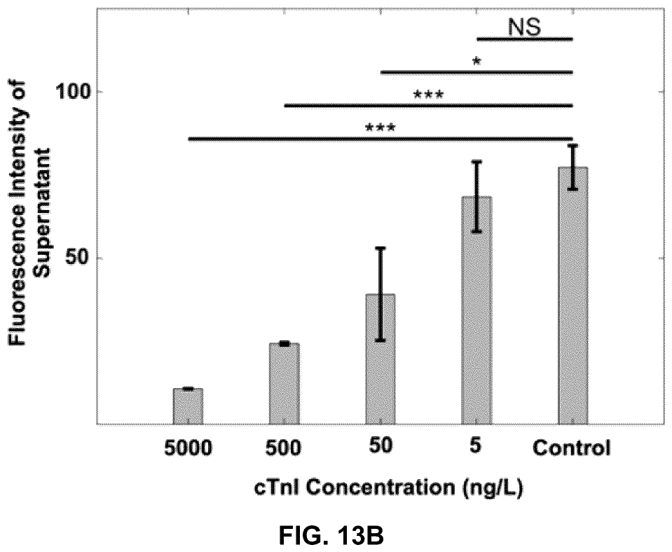

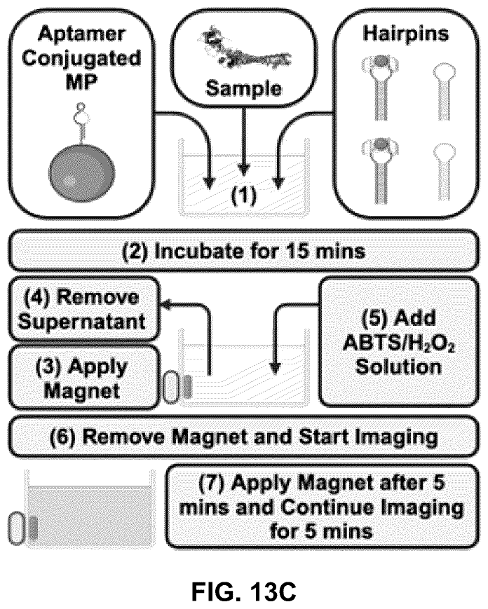

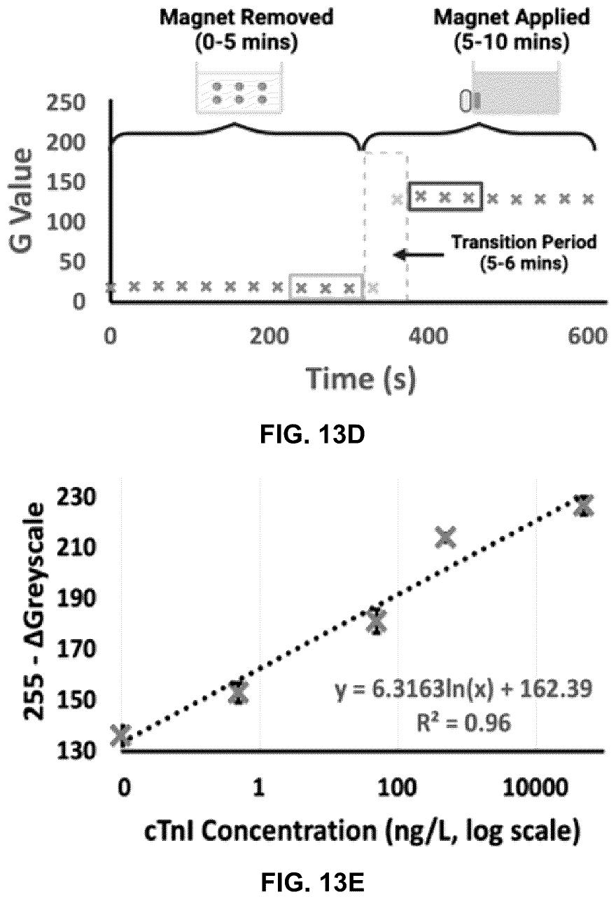

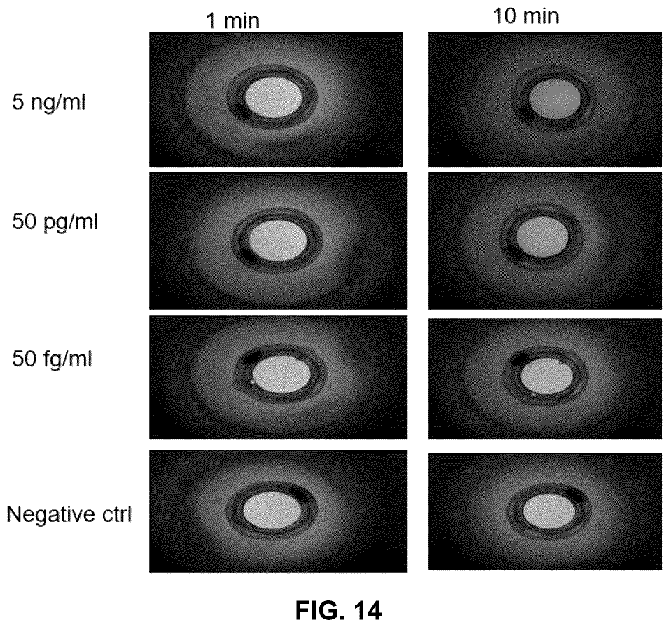

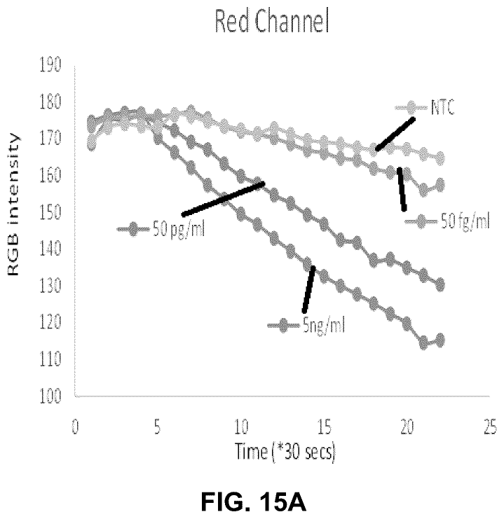

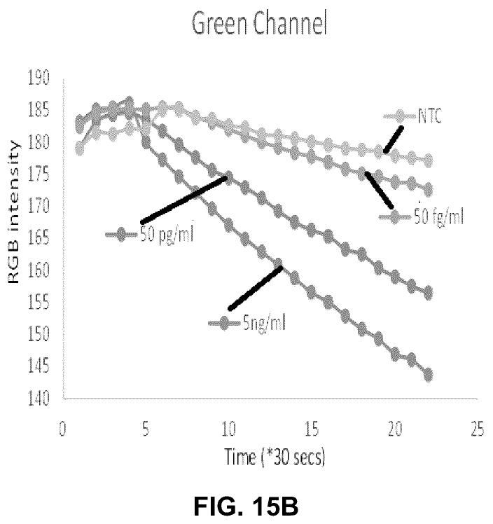

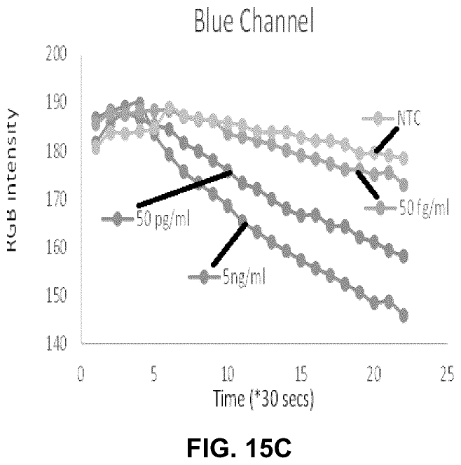

A schematic of the colorimetric assay approach is illustrated in FIG. 12A. While the MP functionalized with the modified Tro4 aptamer for capturing cTnl remains unchanged, the number of hairpins used for HCR amplification has been increased from 2 to 4. Two of these hairpins, HTG-H1 and HTG-H2, contain a hemin-incorporated trimeric G-quadruplex DNAzyme complex (FIG. 12A (3)). In a previous assay using 2 hairpins, HCR amplification proceeded in a single direction. In this revised assay, the hairpins were designed to promote hyperbranched chain formation, allowing branches to form off the main chain (FIG. 13A (4)). It was hypothesized that this bidirectional amplification approach would help reduce the total assay time. Furthermore, the hemin/trimeric G-quadruplex DNAzyme complex is significantly reduced in size compared to the SERS-active nanoparticles and provides an efficient catalyst for the turnover of ABTS²⁻ to ABTS⁻ in the presence of hydrogen peroxide (H₂O₂) resulting in generation of a green solution (FIG. 12B). Using this improved approach, cTnl levels can be quantified by monitoring the extent of the catalyzed color change. To facilitate this, a small, low-cost imaging system was built, as shown in FIG. 12C. The assay is conducted in a small cylindrical flat-bottomed sample tube, which is inserted into a 3D-printed holder. A collar embedded with a small, rectangular N52 magnet that can be slipped over the neck of the holder to magnetically separate the MPs was also designed. The holder is mounted onto a sample plate with an LED backlight positioned beneath it to transmit light through the sample tube. A simple circuit, consisting of a switch, rechargeable battery, and variable resistor for adjusting the LED backlight's luminosity, is housed within an electronics enclosure. Images of the backlit sample are captured using a manual focus webcam mounted in a 3D-printed lid, with the aperture suspended directly above the sample tube. Alignment and blocking of external light are ensured by magnetically coupling the lid to the sample plate. Images are collected from the assay at user-specified intervals (usually every 30 seconds) using a custom Python program operated via a graphical user interface. The program saves the images to a specified directory and provides real-time visualization of R, G, and B values by averaging approximately 300 extracted RGB values from the center of the sample vial. Example images collected by the imaging system at the start (t=0 s) and end (t=600 s) of an assay can be seen at the bottom of FIG. 12B. The red rings in the center of the images indicate the area where the RGB values of the pixels are extracted and averaged. Once imaging is complete, the program exports a text file containing timestamps and the average R, G, and B values for further analysis.

### Refinement and Validation of the Hyperbranched HCR Approach for Ctnl Testing

Before purchasing the hairpin sequences used to facilitate the hyperbranched HCR amplification, over 100 candidates were screened through simulation studies in NUPACK. This allowed identification of sequences that minimized unwanted cross-hybridization in the absence of the modified Tro4 aptamer and cTnl. Simulation results for the top 4 candidates capable of facilitating the hyperbranched HCR amplification mechanism with minimal side product formation is shown in FIG. 13A. The results demonstrate that when the hairpins HTG-H1, H1, HTG-H2, and H2 are combined at a high concentration (1000 nM) at 25° C., only three side products, indicated by (2), (3), and (4), are formed in varying concentrations. Importantly, in the absence of the modified aptamer and cTnl, the concentration of the HTG-H1, which binds to the initiator on the conformationally changed aptamer bound to cTnl, remains at 1000 nM suggesting it stays closed (1). This ensures that none of the side products become tethered to the magnetic particle (MP) and can be easily removed via washing. Consequently, this significantly limits their contribution to non-specific colorimetric signal generation. Fluorescence studies were conducted to evaluate whether HTG-H1 hybridizes with the initiator sequence of the modified Tro4 aptamer-conjugated MPs in the presence of varying concentrations of cTnl. Fluorescently tagged HTG-H1, cTnl, and modified Tro4 aptamer-conjugated MPs were incubated together for 15 minutes. After incubation, the MPs were magnetically separated, and the fluorescence signal from the supernatant was measured. The results, shown in FIG. 13B, indicate that the fluorescence intensity of the supernatant inversely correlates with the concentration of cTnl. Specifically, higher concentrations of cTnl result in more HTG-H1 hybridizing to the aptamer initiator sequence, thereby reducing the concentration of fluorescently tagged HTG-H1 in the supernatant. This demonstrates that the conformational change in the aptamer in the presence of cTnl exposes the initiator sequence, allowing it to hybridize with HTG-H1, a crucial event for HCR amplification to proceed. However, it is notable that the control sample (no cTnl present) could not be distinguished from the lowest measured cTnl concentration (0.5 ng/mL), further emphasizing the need for HCR amplification to achieve the sensitivity required for 4th generation hs-cTnl detection. The protocol used for preliminary colorimetric cTnl testing studies is illustrated in FIG. 13C. Briefly, the aptamer-conjugated magnetic particles (MPs) and hairpins (a combined volume of 25 μL in a pre-optimized high salt concentration assay buffer) are mixed with the cTnl sample (25 μL) in a sample tube (FIG. 12C). This mixture is then placed onto the sample platform of the imaging system (FIG. 12C) and incubated for 15 minutes. After incubation, the MPs are magnetically separated, and the supernatant is removed and replaced with ABTS²⁻/H₂O₂ solution. Imaging begins immediately at this stage. The magnet is removed to allow the MPs to disperse within the ABTS²⁻/H₂O₂ solution, increasing the surface area for catalysis. A 5-minute dispersion period was found to provide the optimal balance between assay time and the differentiation of cTnl at low concentrations. The magnet is then reapplied, pulling the MPs aside to allow the extent of the green color change to be observed. Imaging continues for an additional 5 minutes.

An example real-time time-series plot displayed on the GUI shows the change in the averaged G channel value extracted from images collected every 30 seconds over a 10-minute period, as presented in FIG. 13D. During the first 5 minutes, when the magnet is removed and the MPs are dispersed within the ABTS²⁻/H₂O₂ solution, the measured G values are low due to the dark color of the MPs reducing light transmission through the test sample. Once the magnet is reapplied, there is a transition period (highlighted in yellow) as the MPs migrate towards the magnet. After the migration is complete, around 6 minutes, there is a noticeable increase in the measured G value. For each assay, the R, G, and B channels are extracted and averaged for three time points on either side of the transition period: 240, 270, and 300 seconds before the transition period (x̅B, highlighted by the orange box) and 390, 420, and 450 seconds after the transition period (x̅A, highlighted by the red box). The change in greyscale value (Δ_{Greyscale}) is calculated by subtracting x̅B from x̅A for each channel and then converted to greyscale using the NTSC formula: 0.299×(R)+0.587×(G)+0.114×(B). Finally, the Δ_{Greyscale} values are subtracted from 255 to generate a direct relationship across a wide dynamic range of cTnl concentrations, as shown in FIG. 13E. Using this aptamer-based hyperbranched HCR approach for cTnl quantification using a low-cost, small form factor imaging/reader system, the limit of detection (LOD) has been calculated to be 0.117 ng/mL, which is competitive with 4th generation hs-cTnl immunoassays performed using sophisticated instrumentation in hospital laboratories. Additionally, the assay is completed in 23 minutes using only 25 μL of sample, which is consistent with current 3rd generation cTnl testing platforms such as the QuidelOrtho-Triage MeterPro.

It should be emphasized that the above-described embodiments of the present disclosure are merely possible examples of implementations set forth for a clear understanding of the principles of the disclosure. Many variations and modifications may be made to the above-described embodiment(s) without departing substantially from the spirit and principles of the disclosure. All such modifications and variations are intended to be included herein within the scope of this disclosure and protected by the following claims.

### REFERENCES

1. Dirks, R. M. et al., “Triggered amplification by hybridization chain reaction,” Proc. Natl. Acad. Sci. U.S.A, vol. 101, no. 43, pp. 15275-15278, October 2004, doi: 10.1073/pnas.0407024101.
2. Heller, G. V. et al., “Plasma beta-endorphin levels in silent myocardial ischemia induced by exercise,” Am. J. Cardiol., vol. 59, no. 8, pp. 735-739, April 1987, doi: 10.1016/0002-9149(87)91083-6.
3. Jo, H. et al., “Electrochemical aptasensor of cardiac troponin I for the early diagnosis of acute myocardial infarction,” Anal. Chem., vol. 87, no. 19, pp. 9869-9875, October 2015, doi: 10.1021/acs.analchem.5b02312.
4. Mitsakakis, K. et al, “Detection of multiple cardiac markers with an integrated acoustic platform for cardiovascular risk assessment,” Anal. Chim. Acta, vol. 699, no. 1, pp. 1-5, August 2011, doi: 10.1016/j.aca.2011.05.016.
5. Ragusa, R. et al., “Epigenetic Regulation of Cardiac Troponin Genes in Pediatric Patients with Heart Failure Supported by Ventricular Assist Device,” Biomedicines, vol. 9, no. 10, p. 1409, October 2021, doi:10.3390/biomedicines9101409.
6. Rani, S. et al., “Superparamagnetism and metamagnetic transition in Fe3O4 nanoparticles synthesized via co-precipitation method at different pH,” Phys. B Condens. Matter, vol. 472, pp. 66-77, September 2015, doi:10.1016/j.physb.2015.05.016.
7. Rezaee, M. A. et al., “Selection of specific inhibitor peptides in enzyme-linked immunosorbent assay (ELISA) of cardiac troponin I using immuno-dominant epitopes as competitor,” J. Immunoassay Immunochem., vol. 38, no. 1, pp. 72-81, 2017, doi:10.1080/15321819.2016.1216444.
8. Schiestel, T. et al., “Controlled surface functionalization of silica nanospheres by covalent conjugation reactions and preparation of high density streptavidin nanoparticles,” J. Nanosci. Nanotechnol., vol. 4, no. 5, pp. 504-511, May 2004, doi:10.1166/jnn.2004.079.
9. Zhang, Y. et al., “A highly sensitive and versatile fluorescent biosensor for pathogen nucleic acid detection based on toehold-mediated strand displacement initiated primer exchange reaction,” Anal. Chim. Acta, vol. 1221, p. 340125, August 2022, doi: 10.1016/j.aca.2022.340125.

What is claimed is:

1. A method for detecting a biomarker in a clinical sample, the method comprising:
(a) binding an aptamer locked in a first conformation to a magnetic nanoparticle (MNP);
(b) contacting the magnetic nanoparticle and aptamer with a clinical sample;
wherein presence of the biomarker in the clinical sample causes the aptamer to undergo a conformational change to a second conformation;
wherein the conformational change results the opening of an initiator which triggers a hybridization chain reaction with a trimeric triplex DNAzyme sequence incorporating a first hairpin and a second hairpin, wherein the first hairpin and the second hairpin are partially complementary to each other, forming a structurally stabilized catalytic trimeric triplex;
(c) contacting the structurally stabilized catalytic trimeric triplex with one or more reagents to produce a detectable signal; and
(d) detecting the signal.

2. The method of claim 1, wherein the biomarker comprises a peptide, a protein, DNA, RNA, a carbohydrate, a lipid, a bacterium, a parasite, or a virus.

3. The method of claim 2, wherein the biomarker comprises cardiac troponin I.

4. The method of claim 1, further comprising contacting the magnetic nanoparticle, aptamer, and clinical sample with a third hairpin and a fourth hairpin.

5. The method of claim 4, wherein performing the method forms a hyperbranched chain connected to the MNP, wherein the hyperbranched chain produces an increased signal intensity relative to an unbranched chain.

6. The method of claim 1, wherein the magnetic nanoparticle comprises iron oxide.

7. The method of claim 6, wherein the iron oxide is co-precipitated with silica and wherein the silica is amino functionalized using (3-aminopropyl)triethoxysilane (APTES).

8. The method of claim 7, wherein the magnetic nanoparticle comprises a streptavidin coating, and wherein the streptavidin coating is conjugated to the APTES.

9. The method of claim 8, wherein the aptamer is conjugated to biotin, and where the biotin binds to the streptavidin coating.

10. The method of claim 1, wherein the one or more reagents comprise hemin, hydrogen peroxide, 3,3′,5,5′-tetramethylbenzidine (TMB), or any combination thereof, and a substrate, wherein the hemin induces stabilization of the catalytic trimeric triplex, and wherein the stabilized catalytic trimeric triplex has horseradish peroxidase activity in the presence of the hydrogen peroxide and the substrate.

11. The method of claim 1, wherein the detectable signal comprises a fluorescence signal, a color, an electrochemical signal, a Raman signal, or another signal.

12. The method of claim 10, wherein the substrate comprises 2,2′-azinobis-(3-ethylbenzothiazoline-6-sulfonic acid) (ABTS), and wherein the horseradish peroxidase activity produces a color in the presence of the hydrogen peroxide and the ABTS.

13. The method of claim 11, wherein an intensity of the color is directly proportional to an amount of biomarker present in the clinical sample.

14. The method of claim 11, wherein a change in intensity of the color is proportional to an amount of biomarker present in the clinical sample.

15. The method of claim 1, wherein the one or more reagents comprise a gold particle, and wherein the detectable signal comprises fluorescent or Raman scattering, and wherein the gold particle comprises a gold nanostar or a gold nanosphere.

16. The method of claim 15, wherein the gold particle comprises a silica shell and a plurality of dye molecules, wherein the dye molecules are encapsulated within the silica shell adjacent to a surface of the gold particle.

17. The method of claim 16, wherein the dye molecules comprise NIR-780.

18. The method of claim 1, wherein the method has a limit of detection (LoD) of about 10 femtomolar.

19. A method for identifying myocardial infarction, the method comprising performing the method of claim 1 on a clinical sample from a subject, wherein the biomarker is cardiac troponin I.

20. The method of claim 19, wherein the myocardial infarction is asymptomatic.
